(19) United States
(12) Patent Application Publication
(10) Pub. No.: US 2004/0234588 A1
(43) Pub. Date: Nov. 25, 2004

(54) Artificial lipoprotein carrier system for bioactive materials

(75) Inventors: Donghao Robert Lu, Chester Springs, PA (US); Mohannad Shawer, San Diego, CA (US)

Correspondence Address: Henry D. Coleman, 714 Colorado Avenue, Bridgeport, CT, 06605, US

(73) Assignee: UNIVERSITY OF GEORGIA RESEARCH FOUNDATION, INC., Athens, GA

(21) Appl. No.: 10/803,562

(22) Filed: Mar. 18, 2004

Related U.S. Application Data
(63) Continuation-in-part of application No. 09/961,028, filed on Sep. 21, 2001.
(60) Provisional application No. 60/234,141, filed on Sep. 21, 2000.
(60) Provisional application No. 60/455,915, filed on Mar. 19, 2003.

(51) Int. Cl. A61K 9/127

(52) U.S. Cl. 424/450

(57) ABSTRACT

The present compositions comprise a microemulsion as a carrier for a bioactive agent, especially a polynucleotide, for example, DNA or RNA, said microemuslion comprising a lipid core which is surrounded by a monolayer of at least one amphipathic lipid and at least one lipidized polymer, the lipidized polymer preferably comprising a lipidized protein or polypeptide in combination with a bioactive agent which is dissolved within or dispersed into one or more of the lipid core or the amphipathic lipid or which is associated with the surface of the microemulsion.

### RELATED APPLICATIONS

This application is a continuation-in-part application of application Ser. No. 09/961,028, filed Sep. 21, 2001 entitled “Artificial Lipoprotein Carrier System for Pharmaceutical Use” and claims the benefit of priority from provisional application serial No. 60/455,915, filed Mar. 19, 2003, of same title, both of which applications are incorporated by reference in their entirety herein.

### FIELD OF THE INVENTION

The present invention relates to novel carriers for bioactive agents which mimic native lipoprotein, preferably, low density lipoprotein.

### BACKGROUND OF THE INVENTION

One of the prerequisites for the action of a drug generally is its ability to penetrate lipid cell membranes. But in order to do this the drug must generally act through its undissociated, lipid soluble part. This chemistry, however, conflicts with the chemistry associated with drug dissolution and its ability to be administered orally or even parenterally, as a drug has to be dissolved in gastric juices in the case of oral administration or a physiological vehicle in the case of parenteral administration, which vehicle normally is an isotonic aqueous solution. Thus, many drugs cannot be administered orally or parenterally unless the drugs can be modified chemically to provide greater dissolution for administration.

Many pharmaceutically active agents now in common use often require formulation compromises in order to prepare the marketed product. In the administration of pharmacologically active agents, it has generally been necessary to use water-soluble agents or to transform the agents into a water-soluble form, so that dissolution properties can be obtained which are appropriate for administration. Thus, many parenteral compositions must be prepared using the salt of the parent compound and an excessive pH. The use of the agents in a water-soluble form, however, has often had several disadvantages. For instance, the excessive pH required for aqueous solutions may often cause side effects. Also, it may sometimes be difficult to attain a desired effect, as the solutions may not be tolerated by the patient.

The instability of many useful drugs and other useful medical compositions poses other formulation problems. At present, emulsions, microemulsions and liposomes constitute the principal approaches to these problems. While such dosage forms are an advance over older forms, they are often associated with erratic bioavailability and instability of their own.

### OBJECTS OF THE INVENTION

It is an object of the present invention to provide novel formulations for the delivery of bioactive agents.

It is an additional object of the invention to provide novel vehicles which may readily deliver lipophilic (hydrophobic) bioactive agents to a patient without the need to reformulate the agents into more water soluble forms.

It is still another object of the invention to provide novel formulations which mimic lipoproteins such that water-insoluble bioactive agents may be solubilized and delivered to the patient through the LDL pathway.

It is yet a further object of the invention to provide formulations which further comprise proteins or polymers which allow for targeting of the formulations to specific organs or tissues within the patient.

It is still another object of the invention to provide methods for enhancing the bioavailability of bioactive agents using compositions according to the present invention.

It is still another object of the invention to provide provide compositions which can be used to deliver or transfect DNA into cells.

One or more of these and/or other objects of the present invention may be readily gleaned from a description of the invention which follows.

### BRIEF DESCRIPTION OF THE DRAWINGS

FIGS. 1 and 2 depict graphs showing the distribution of components of the lipid structure of PSME and location of BCH in the formulation which were assessed based on experiments set forth in example 1. Density gradient ultracentrifugation fractionated the emulsion into 3 particle size populations with structures and compositions resembling native lipoproteins. See table of example 1. The graph depicts where the different components are located within the PSME.

FIG. 3 depicts the mass spectrum of the modified lysozyme from example 4 which showed an obvious increase in the molecular weight of the lysozyme. FIGS. 4 and 5 depict visual representations of the artificial LDL's for Bio-targeting and for Gene delivery according to the present invention.

FIG. 6 shows the size change of nanoemulsion particles upon storage at room temperature

FIG. 7 depicts agarose gel electrophoresis of nanoemulsion particles and their complexes with p-PLL stained with Nile Red (Lane 1: nanoemulsion; Lane2 to Lane 5 were the complex of nanoemulsion and p-PLL with the ratio of p-PLL to triolein to be 0.125:1, 0.25:1, 0.5:1 and 1:1, respectively)

FIG. 8 depicts the agarose gel electrophoresis of the complex of nanoemulsion and p-PLL with plasmid DNA stained with ethidium bromide (Lane 1 and Lane 8: Pure DNA; Lane 2: DNA/p-PLL; Lane 3 to Lane 7 were complexes of nanoemulsion with different amount of p-PLL and DNA. The ratio of p-PLL to triolein was 1:1, 1:0.5, 1:0.25, 1:0.125, and 1:0.0625, respectively).

FIG. 9 depicts the zeta potential of the nanoemulsion particles and their complexes with different amount of p-PLL and DNA (2 μg)

FIG. 10 depicts the mobility of the nanoemulsion particles and their complexes with different amount of p-PLL and DNA (2 μg)

FIG. 11 depicts X-Gal staining of glioma cells (A: Cells transfected using Lipofectamine™ reagent; B: Control; C: Cells transfected using nanoemulsion/p-PLL/DNA complex)

FIG. 12 shows the effect of chloroquine on the transfection by the nanoemulsion gene delivery system (white bar—untreated with chloroquine; gray bar—treated with chloroquine)

FIG. 13 depicts a cytotoxicity comparison between nanoemulsion/p-PLL and Lipofectamine: control (♦, top), nanoemulsion/p-PLL (□, middle) and Lipofectamine (▾, bottom).

FIG. 14 depicts (A) Artificial lipoproteins (20-100 nm) and (B) natural human lipoproteins (those in 20-100 nm range)

FIG. 15 shows the Percentage transfection efficiency in human glioma SF-767 cell line with the artificial lipoprotein formulation and lipofectamine 2000. Lane 1 (top) is the negative control (NE/p-PLL/pCDNA-3). Lanes 2 (L0.5) and 3 (L1) are the positive controls (p-PLL/pCDNA-RG) and the quantity of p-PLL added was equivalent to that in Lanes 10 and 11, respectively. Lanes 4, 5, 6 and 7 refer to the transfection using lipofectamine 2000 with pCDNA-RG/lipofectamine ratio of 1:0.5, 1:2, 1:3 and 1:5, respectively (pCDNA-RG quantity was kept constant). Lanes 8, 9, 10 and 11 refer to the transfection using artificial lipoprotein system (NE/p-PLL/pCDNA-RG) with the p-PLL to triolein ratio of 0.125:1, 0.25:1, 0.5:1 and 1:1, respectively (pCDNA-RG quantity was kept constant). (n=3 for all experiments).

### BRIEF SUMMARY OF THE INVENTION

The present invention relates to novel carriers for bioactive agents, preferably, hydrophobic drugs for enhanced delivery of the bioactive agents to patient. Compositions according to the present invention comprise a microemulsion which contains a bioactive agent, said microemulsion comprising a lipid core (such as a triglyceride) which is preferably neutral in pH, stabilized by a monolayer of an amphipathic lipid, preferably a phospholipid and at least one bioactive agent which, depending upon the chemical characteristics of the bioactive agent, may be dissolved or dispersed within the lipid core, the amphipathic lipid monolayer, or even the surface of the microemulsion (especially when the bioactive agent is a macromolecule such as a polynucleotide such as DNA). Compositions according to the present invention also comprise at least one lipidized polymer, preferably a lipidized protein or polypeptide, wherein at least a portion of said lipidized polymer is dissolved or dispersed within the amphipathic lipid monolayer and/or core lipid and at least a portion of the polymer is associated with the surface of the microemulsion.

Compositions according to the present invention may optionally comprise additional components such as fatty acids, steroid compounds and other compounds which may be added to the amphipathic lipid monolayer to modify the chemical and/or physical characteristics of the microemulsion.

Carriers according to the present invention may be used to mimic naturally occurring or native lipoprotein. The present approach may be used to accommodate large numbers of bioactive agents of varying physicochemical characteristics which are difficult to administer with acceptable bioavailability and pharmacokinetics. The present carriers may be used to dramatically increase the bioavailability of a desired agent and in certain embodiments, to enhance delivery of agents to a specific site of activity within the patient.

The present compositions therefore comprise a microemulsion as a carrier for a bioactive agent, said microemuslion comprising a lipid core which is surrounded by a monolayer of at least one amphipathic lipid and at least one lipidized polymer, the lipidized polymer preferably comprising a lipidized protein or polypeptide in combination with a bioactive agent which is dissolved within or dispersed into one or more of the lipid core or the amphipathic lipid or which is associated with the surface of the microemulsion. The bioactive agent may be any agent which exhibits a biological or pharmacological effect in a biological system, and preferably is a drug to be delivered to treat a condition or disease in a patient. More preferred bioactive agents include hydrophobic neutral or amphipathic drugs, especially those which may be more difficult to deliver because of their hydrophobic properties which severely limits water solubility, even more preferably anti-cancer or neoplastic agents.

Methods for enhancing the bioavailability of bioactive agents generally, and in particular, to specific sites within the patient using the compositions according to the present invention, represents an additional aspect of the present invention.

### DETAILED DESCRIPTION OF THE INVENTION

The following terms shall be used throughout the specification to describe the present invention.

The term “microemulsion” as used herein shall refer to an oil in water emulsion system with a small size (generally, having a median particle diameter in the sub-micron range, preferably ranging in size from about 1 to about 950 nanometers in diameter, more preferably about 5 to about 750 nanometers in diameter, even more preferably about 10 to about 500 nanometers in diameter, even more preferably about 25 to about 150 nanometers in diameter) which resembles the emulsion droplet in a natural or native lipoprotein, preferably a low density lipoprotein. The size of the microemulsion will generally depend upon the amphipathic lipid used to form the monolayer which surrounds the lipid core as well as the relative concentration of lipid in the lipid core to amphipathic lipid which surrounds the lipid core. The microemulsion according to the present invention is generally a spherical particle and is similar in size and shape to the lipid portion of a natural lipoprotein. The term “carrier” shall refer to the microemulsion which preferably includes a lipidized polymer, but excludes the bioactive agent. Note that the use of the terms “microemulsion”, “submicron emulsion”, “phospholipid submicron emulsion”, “lipoprotein-resembling submicron emulsion” and lipoprotein-resembling particles” are synonymous terms used throughout the specification to describe emulsion system according to the present invention.

The term “bioactive agent” is used throughout the specification to describe a chemical compound which produces a biological effect in a biological system, preferably a mammalian system. Bioactive agents include drugs, toxins, pesticides, nucleic acids, including DNA in any form, including naked DNA and RNA, including transfer RNA, proteins and any number of compounds which produce a pharmacological response or other biological response in a patient. Preferably, bioactive agents according to the present invention include any one or more compounds which are drugs and which may be used to treat a condition or disease state in a patient, in particular, a human patient, or alternatively may comprise nucleic acid, such as a polynucleotide including DNA or RNA, preferably non-native (non-autologous) including naked DNA to elicit a biological response. Bioactive agents include a broad range of compounds including, for example, anesthetics, systemic antibiotics, antiparasitics, systemic quinolones, anti-infectives, anti-inflammatories, aminoglycosides, cephalosporins, penicillins, antidotes, anti-cholinesterases, metal poisoning antidotes, antineoplastics, 5′-fluorouracil, cytotoxic agents, hormones, steroids, immunomodulators, cytokines, systemic antivirals, systemic antifungals, biologicals, alpha-antitrypsin, bone metabolism regulators, hypercalcemic agent, cardiovascular agents, beta blockers, cerebral vasodilators, cerebral metabolic enhancers, cholinesterase inhibitors, colony stimulating factors, granulocyte-colony stimulating factors, granulocyte macrophage-colony stimulating factors, vasopressors, local diabetic agents, diagnostics such as CT scan enhancers and angiocardiography agents, adenosine deaminase deficiency agents, gonadotropin inhibitors, adrenal cortical steroid inhibitors, gonadotropin releasing hormone stimulant, urofollitropins, muscle relaxants such as neuromuscular blocking agents, prostaglandin analogs, prostaglandins, prostaglandin inhibitors, respiratory therapy agents, anticholinergics, beta andrenergic stimulators, sympathomimetics, and thrombolytics, antithrobotics, anticoagulants, antibiotics antiplatelet agents, thrombolytics, antiproliferatives, steroidal and nonsteroidal antiinflammatories, agents that inhibit hyperplasia and in particular restenosis, smooth muscle cell inhibitors, growth factors, growth factor inhibitors, cell adhesion inhibitors, cell adhesion promoters and drugs that may enhance the formation of healthy neointimal tissue, including endothelial cell regeneration agents, clonidine, estradiol, nicotine, nitroglycerin, and scopolamine, all of which are commercially available in the form of transdermal delivery devices. Others include antiinflammatory drugs, both steroidal (e.g., hydrocortisone, prednisolone, triamcinolone) and nonsteroidal (e.g., naproxen, piroxicam); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotazoals (e.g., metronidazole); antifungals (e.g., nystatin); coronary vasodilators (e.g., nitroglycerin); calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors and angiotensin converting enzyme inhibitors (e.g., captopril, lisinopril); other antihypertensives (e.g., propranolol); leukotriene antagonists (e.g., ICI 204,219); anti-ulceratives such as H2 antagonists; steroidal hormones (e.g., progesterone, testosterone, estradiol, levonorgestrel); antivirals and/or immunomodulators (e.g., 1-isobutyl-1H-imidazo[4,5-c]quinolin-4-amine, 1-(2-hydroxy-2-methylpropyl)-1H-imidazo[4,5-c]quinoline-4-amine, and other compounds disclosed in U.S. Pat. No. 4,689,338, incorporated herein by reference, acyclovir); local anesthetics (e.g., benzocaine, propofol); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, fentanyl); peptide hormones (e.g., human or animal growth hormones, LHRH); cardioactive products such as atriopeptides; proteinaceous products (e.g., insulin); enzymes (e.g., anti-plaque enzymes, lysozyme, dextranase); antinauseants (e.g., scopolomine); anticonvulsants (e.g., carbamazine); immunosuppressives (e.g., cyclosporine); psychotherapeutics (e.g., diazepam); sedatives (e.g., phenobarbital); hypnotics; anticoagulants (e.g., heparin); analgesics (e.g., acetaminophen); antimigraine agents (e.g., ergotamine, melatonin, sumatriptan); antiarrhythmic agents (e.g., flecainide); antiemetics (e.g., metaclopromide, ondansetron); anticancer agents (e.g., methotrexate); neurologic agents such as anxiolytic drugs; hemostatics; anti-obesity agents; antigout agents; antianxiety agents; antiinflammatory agents; hormones; immunosuppressive agents; hyplipedmic agents; antiparkinson agents; antifungal agents; analgesics; antimanic agents; antipyretics; antiarthritic agents; antiplatetet agents; anticonvulsants; antidiabetic agents, anticoagulants, antiarrhythmics, antianginal agents; and the like, as well as pharmaceutically acceptable salts, esters, solvates and clathrates thereof., among numerous others.

Bioactive agents preferably comprise at least about 0.001% to about 50%, preferably about 0.05% to about 50% by weight of the final composition (which weight includes the carrier, lipidized polymer and drugs in the final composition) according to the present invention, more preferably about 1% to about 35% by weight of the final composition. In certain preferred aspects according to the present invention, an amount of drug ranging from about 1% to about 15% by weight of the final composition is preferred. The amount of bioactive agent incorporated into the final composition will reflect the componentry of the microemulsion, the lipidized polymer used (which amount may vary greatly in the present compositions), the effective amount of agent required to elicit its intended effect in the patient. One of ordinary skill will be able to readily vary the componentry of the microemulsion, the amount and type of lipidized polymer in conjunction with the physicochemical properties of the bioactive agent in providing compositions according to the present invention.

The term “nucleic acid” shall mean DNA or RNA in any form, whether in a naked form “naked DNA” or incorporated into a vector for purposes of expressing a protein.

The term “patient” is used throughout the specification to describe an animal, preferably a mammal and especially a human, to whom treatment, including prophylactic treatment, with the pharmaceutical compositions according to the present invention is provided.

The term “effective amount” is used throughout the specification to describe concentrations or amounts of composition according to the present invention which may be used to produce a favorable change in a disease or condition treated or to otherwise effect a biological, including a pharmacological, result. With respect to individual components which may comprise the final compositions according to the present invention, the term effective amount is used to describe that amount of a component which are included in compositions according to the present invention in order to produce an intended effect.

The term “amphipathic lipid” as used herein shall mean any suitable material of lipid or of a hydrophobic nature, preferably a phospholipid which can be used to create a monolayer which surrounds the lipid core in microemulsions according to the present invention. Generally, an amphipathic lipid has a hydrophobic neutral portion at one end of the molecule and a hydrophilic, often charged (often, anionic) portion at the other end of the molecule. In certain preferred aspects according to the present invention which relate to the delivery of DNA, the inclusion of positively charged (cationic) lipids, with or without lipidized protein or polymer, may aid in the delivery of DNA using the microemulsion compositions according to the present invention. In the present invention, the amphipathic lipid produces a monolayer such that a hydrophobic portion of the amphipathic lipid material orients toward the interior of the microemulsion or lipid core, while a hydrophilic portion orients toward the aqueous phase or surface of the microemulsion. Lipids at the surface of the microemulsion may be charged (positive or negative) or neutral. Although any number of amphipathic lipids can be used in the present invention, including for example, the sodium and potassium salts of fatty acids, preferred amphipathic lipids include the phospholipids such as phosphatidylcholine (PC), cephalin, isolecithin (lysophosphatidyl choline), phosphatidylethanolamine (PE), distearoylphosphatidylcholine (DSPC), phosphatidylserine (PS), phosphatidylglycerol (PG), phosphatidic acid (PA), phosphatidylinositol (PI), sphingomyelin (SPM), and the like, alone or in combination. The phospholipids can be synthetic or derived from natural sources such as egg or soy. Some synthetic phospholipids which can be used in the present invention include, for example, dimyristoylphosphatidylcholine (DMPC) and dimyristoylphosphatidylglycerol (DMPG). The phospholipids which may be used in the present invention include chemical modified, conjugated or polymerized phospholipids such as pegylated phospholipids (which contains polyethylene glycol chains).

The amount of phospholipid which can be used in compositions according to the present invention is that amount effective to form a microemulsion in combination with lipid and, in most instances, bioactive agent. Preferably, the amount of phospholipid ranges from about 3% to about 75% by weight, more preferably about 15% to about 65% by weight of the microemulsion (i.e., excluding the lipidized polymer component) composition which weight includes lipid, phospholipid, optional steroidal component and bioactive agent, but which weight excludes lipidized polymer and any aqueous phase associated with the microemulsion in the final delivered composition. Preferred phospholipids include a mixture of egg phosphatidyl choline (22.7%) and lyosphophosphatidyl choline (2.3%) for a total phospholipid content of about 25% by weight. In addition to phospholipid in the phospholipid monolayer, a steroidal component may also be included in an amount ranging from about 0% to about 25% by weight of the phospholipid content of the monolayer, preferably, at least about 0.05% within this range. The amount of lipidized polymer which may be included in final compositions according to the present invention ranges from about 0.01% to about 65% by weight or more, preferably about 0.5% to about 50% by weight, even more preferably about 1% to about 35% by weight and even more preferably about 2.5% to about 15-20% by weight, depending on the polymer used. In a number of instances, where, for example, lysozyme is used, the amount of protein associated with the microemulsion will range from about 3-10% or more by weight, preferably about 5.0-8.0% or more by weight of the final composition. In certain preferred embodiments related to microemulsion compositions according to the present invention which aid in the delivery of DNA, the inclusion or use of positively charged lipids is preferred.

The amphipathic lipid monolayer (as well as the lipid core) may also include steroidal components such as cholesterol, polyethylene glycol derivatives of cholesterol (PEG-cholesterols), coprostanol, cholestanol, or cholestane, or combinations of these steroids. Preferred steroids include cholesterol and cholesterol-like steroids such as cholestane. These steroids may be neutral or charged, especially where they are found at the surface of the microemulsion. It is noted here that certain positively charged steroids included in the formulations with or without lipidized protein or polymer may aid in the delivery of DNA using the microemulsion compositions according to the present invention. In addition, steroid esters (preferably, C₁ to C₂₄ steroid esters, which may be formed by reacting a free hydroxyl group on the steroid with an organic acid, preferably a C₁₂ to C₂₄ fatty acid, or alternatively, by reacting a carboxylic acid group on the steroid with an alcohol, preferably, a C₁₂ to C₂₄ fatty alcohol) such as, for example, cholesterol oleate, among numerous others, may also be included. The steroid compounds may be added in amounts ranging from about 0.005% to about 20% by weight of the amphipathic lipid used to form the monolayer of the microemulsion. Steroid components may be added to the microemulsion primarily to stabilize the microemulsion as well as to change other characteristics of the compositions. The amphipathic monolayer of the present invention may also contain glycolipids.

The term “lipids” or “lipid core” is used throughout the specification to describe a core of the microemulsion which is made from a pharmaceutically acceptable lipid material, which is generally defined as a lubricious and hydrophobic material which is obtained from animal, vegetable or mineral matter or is synthetically prepared. Oils for use in the present invention are generally neutral (i.e., uncharged) and are generally neither acidic nor basic. Oils for use in the present invention may include petroleum-based oil derivatives such as purified petrolatum and mineral oil (especially when the microemulsions are formulated for use in cosmetic and/or topical applications), but in general, the lipids used in the present invention are preferably mono-, di- and triglycerides or other neutral, esterified fatty acid compounds.

Petroleum-derived lipids or oils for use in the present invention include aliphatic or wax-based oils and mixed base oils and may include relatively polar and non-polar oils. “Non-polar” oils are generally oils such as petrolatum or mineral oil or its derivatives which are hydrocarbons and are more hydrophobic and lipophilic compared to certain natural and synthetic oils, such as mono-, di- or triglycerides or other esters, which may be referred to as “polar” oils. It is understood that within the class of lipids, that the use of the terms “non-polar” and “polar” are relative within this very hydrophobic and lipophilic class, and all of the oils tend to be much more hydrophobic and lipophilic than the hydrophilic portion of the amphipathic lipids which are also used in the present invention.

In addition to the above-described oils, certain pharmaceutically acceptable essential oils derived from plants such as volatile liquids derived from flowers, stems and leaves and other parts of the plant which may include terpenoids and other natural products including triglycerides may also be considered lipids for purposes of the present invention. The use of naturally occurring triglycerides as lipids for use in the present invention is clearly preferred.

Preferred lipids or oils for use in the present invention may include, for example, mono-, di- and triglycerides which may be natural or synthetic (derived from esterification of glycerol and at least one or more saturated or unsaturated organic acid, such as, for example, butyric, caproic, palmitic, stearic, oleic, linoleic or linolenic acids, among numerous others, preferably a fatty organic acid, comprising between 8 and 26 carbon atoms). Glyceride esters for use in the present invention include vegetable oils derived chiefly from seeds, nuts and vegetables and include drying oils, for example, linseed, iticica and tung, among others; semi-drying oils, for example, soybean, sunflower, safflower, palm, poppy seed, corn, sesame seed, olive, canola and cottonseed oil; non-drying oils, for example castor and coconut oil, among numerous others. Hydrogenated vegetable oils also may be used in the present invention. Animal oils are also contemplated for use as glyceride esters and include, for example, fats such as tallow, lard and stearin and liquid fats, such as fish oils, fish liver oils and other animal oils, including sperm oil, and vitamins such as the tocopherols, among numerous others. In addition, a number of other oils may be used, including C₁₂ to C₃₀ (or higher) fatty esters (other than the glyceride esters, which are described above). Triolein is a preferred lipid for use in the present invention.

The ratio of the amount of lipid to drug in the present compositions ranges from about 50:1 to about 1:10, preferably about 20:1 to about 1:2, depending upon the hydrophobicity (lipophilicity) of the bioactive agent to be used. The amount of lipid and drug generally ranges from about 5% to about 85% by weight of the microemulsion (i.e., the composition without the lipidized polymer), preferably about 10% to about 75% by weight. The preferred ratio is approximately 70% by weight of the microemulsion, with triolein as the preferred lipid used.

The lipid core of the microemulsion may contain a steroidal component (preferably, an esterified steroidal component such as cholesterol oleate or a similar ester of cholesterol) in the range of about 0 to about 50% by weight of the core (lipid plus hydrophobic bioactive agent).

The term “lipidized polymer” is used to describe a primarily hydrophilic polymer of varying chemical composition which has been modified (preferably through covalent bonding) to contain fatty acid (generally, a C₈-C₅₂ fatty acid, preferably C₈-C₂₆ fatty acid), phospholipid or other hydrophobic (such as a hydrophobic steroidal component or other hydrophobic moiety) residues in order such that the fatty acid or hydrophobic portion of the lipidized polymer will readily interact with (i.e., dissolve or disperse into) the lipid component of the microemulsion (whether that lipid component is in the lipid core or the lipid portion of the amphipathic lipid monolayer) while the non-lipid component of the polymer will associate with the surface of the microemulsion.

Lipidized polymers according to the present invention are readily formed by reacting fatty acids or phospholipids (or their corresponding activated esters or equivalent groups such as acyl halides) of varying lengths with one or more nucleophilic positions on the polymer, such as a free amine, hydroxyl or thiol containing group, preferably an amine group, which results in formation of an amide. Preferably, the polymer from which the lipidized polymer is prepared is a protein or polypeptide such as polylysine or other polypeptide containing at least 3 amino acid units, and more preferably is a protein or polypeptide which functions to bind to a target at a cell site within the patient or alternatively, functions to bind other components such as other proteins, polypeptides and bioactive agents such as drugs, among others, to be delivered by the microemulsion.

Alternatively, lipidized polymer (preferably, protein) may also be carried out at the surface of the preformed microemulsion system by using reactive phospholipid (an activated form of phospholipid wherein the phosphate group is activated, for example, as an activated phosphate ester), cholesterol, fatty acids, or other hydrophobic surfactants while formulating the microemulsion, followed by incubation of the microemulsion with the protein for the reaction to occur.

In the case of the use of protein, in general, the attachment of a fatty or other hydrophobic chain to the protein is very much needed in order to ensure the association between the microemulsion components and the protein.

The preferred chemical lipidization of the protein according to the present invention maintains the biological integrity or function of the protein. Other procedures for lipidization may also be used, including, for example, a reverse micelle system for protein hydrophobization as described by Kabanov et.al (Biol. Mem. Volume 2(10), 1989, pp.1769-1785), conjugation under organic solvent conditions as described by Hashimoto, et.al (Pharm.Res, Vol6, No.2, 1989), conjugation under aqueous solutions using micellar suspension as described by Huang, et. al (Biol.Chem. Vol 255,No.17, pp 8015-8018, 1980).

The method used to prepare compositions according to the present invention depends upon the utilization of a mixture of aqueous and organic solvent (dimethyl formamide) forming a one-phase reaction media to ensure the solubilization of the protein and the fatty acid reagent. One or more of other organic solvents may also be used. The degree of lipidization of the polymer/protein component may also be controlled by the ratio of fatty acid reagent/protein (or other polymer). Numerous proteins and polymers can be lipidized in a similar manner.

Preferably, the polymer is a biological polymer, such as a protein or polypeptide, but also may comprise a polynucleotide (preferably, deoxyribonucleic acid or DNA, because these polymers tend to be less labile than are polymers of ribonucleic acid) of at least three nucleotide units. In general, polymers which are used in the present invention which are other than a protein, polypeptide or polynucleotide are used in cosmetic or topical applications or applications other than for the systemic delivery of a drug. Of course, biodegradable and/or bioresorbable synthetic hydrophilic polymers such as polyesters including polylactic acid and polyglycolic acid polymers, among others, including polyvinylpyrrolidone, may also be used in the present invention in formulations to be used for systemic delivery of drugs. The term “protein” shall mean any protein, regardless of whether that protein is a polypeptide, a glycoprotein or a related protein.

Preferred proteins or polypeptides which may be used in the present invention include, for example, enzymes, cell surface proteins, hormones, antibodies, growth factors, clotting factors, neuroproteins, tumor suppressors, toxins, antigens and epitopes of antigens, apolipoproteins including for example, apolipoprotein B100, apolipotroeint E, endogenous or exogenous tumor antigenic proteins, among numerous others, including bioinvasive molecules (like bacterial invasives), lectins, lectin-like molecules, bacterial toxins (cholera toxin) and macromolecules with bioadhesive properties. Proteins can be chosen to have bio-targeting ability (hereinafter, referred to as “targeting proteins”) in order to direct the microemulsion particle to different biological, especially tissue sites. This approach may dramatically increase the delivery of a bioactive agent to a particular site within a patient in order to enhance therapy, especially where the bioactive agent is seen to have tissue-specific or tissue-selective activity. Preferred targeting proteins or polypeptides include, for example, immunoglobulins or appropriate epitopes, transferrin, avidin, hormones, enzymes, integrin, polylysine, Apolipoprotein E, monoclonal antibodies, and the like. Also appropriate for use in the present invention is any part of the proteins or a synthetic analogue. The preferred reaction conditions are described in detail hereinbelow. Still other preferred proteins include receptors such as hormone receptors including insulin receptors and growth factor receptors, among others. Especially preferred proteins include, for example lysozyme and avidin. In certain preferred embodiments, the inclusion of apolipoprotein B100, apolipoprotein E or apolipoprotein, in combination with one or more targeting proteins such as avidin or lysozyme, may provide a targeting approach to the delivery of drug through the LDL pathway or to other tissues within the body.

In a particularly preferred embodiment according to the present invention, avidin is lipidized using a C₈-C₅₂ fatty acid, preferably a fatty acid containing between eight and 26 carbon atoms, including such fatty acids as palmitic, stearic, oleic, linoleic or linolenic acids, among numerous others. Avidin, a glycoprotein, is preferred in certain instances, because once it is lipidized and formulated into the present microemulsion compositions, it is capable of binding to any biotinylated component and allows a biotinylated component to be attached to the microemulsion through a biotin-avidin complex. The lipidized avidin embodiment of the present invention is especially useful for attaching biotinylated antibodies, as well as a number of biotinylated proteins (such as enzymes, toxins and antigens, among others) as well as biotinylated oligo- and polynucleotides to the microemulsion according to the present invention.

This embodiment is advantageous because it results in a product which can function as a universal station for the attachment of various biotinylated proteins (enzymes, toxins, antibodies, etc.), polymers or drugs. The biotin-avidin embodiment according to the present invention is particularly advantageous approach when attaching an antibody to the microemulsion because it results in standardization, avoids the potential problems of non-selective modification of the antibody and disorientation of the antibody in the microemulsion arising from direct modification on the antibody (potentially resulting in the antibody binding site being buried inside the microemulsion and being unavailable for binding). See atttached FIG. 4.

The synthetic lipoprotein according to the present invention is distinguishable from the natural lipoprotein in that the natural lipoprotein has an apolipoprotein (for example, apoplipoprotein B100 in the case of low density lipoprotein) associated with it, while the lipoprotein according to the present invention has a protein which has been chemically modified to attach hydrophobic groups such as fatty acid chains in order to more closely have the protein associate with the lipid portion of the synthetic lipoprotein. This allows the protein to lower the surface tension at the microemulsion surface and thereof, enhancing the stability of the microemulsion. In the case of the natural low density lipoprotein, it is believed that the association of apolipoprotein B100 with the lipid portion serves as a component for receptor interaction. The protein penetrates the whole natural microemulsion system to enhance the LDL particle stability. This is due to its chemical nature of having a hydrophobic region that spans the core of the LDL microemulsion and a hydrophilic region which protrudes to the surface of the LDL microemulsion which also serves as a searching tool for LDL receptors.

In certain preferred aspects according to the present invention which include a lipidized polymer in effective amounts, the resulting microemulsion exhibits favorable characteristics of enhanced stability, in comparison to similar microemulsions which exclude lipidized polymer, preferably lipidized protein. Enhanced stability within this context shall mean that the resulting microemulsion particles exhibit a consistent size (generally, approximately within the original size estimate without appreciable or substantial change), degradation is substantially reduced and aggregation is substantially reduced or preferably eliminated. These favorable characteristics result in the microemulsion compositions according to the present invention being storage stable in comparison to prior art compositions even in final emulsion form (i.e., when formulated in combination with an aqueous solution prior to administration to a patient).

Another aspect of the present invention relates to a method or methods for transferring an active agent within the microemulsion of the present invention to LDL or other natural lipoprotein (the term “natural lipoprotein” as used herein describing lipoproteins which are found naturally in the patient as opposed to the synthetic lipoproteins which may be used in the present invention), even in the presence of serum proteins. The characteristic of the present invention in this regard is a particularly beneficial and efficient method for transferring bioactive agents of the present invention into LDL or other natural lipoproteins including, for example HDL (high density lipoprotein), VLDL (very low density lipoproteins), IDL (intermediate density lipoproteins) and chylomicrons and results in the agent being delivered to sites within the patient more efficiently than using prior art methods, including liposomes. This method may be used in vitro (generally, in the presence of an effective amount of a lipid transfer protein) to produce natural LDL's or other lipoproteins containing bioactive agents which may subsequently be used to administer bioactive agents to a patient or alternatively, the method may be used in vivo, to enhance the delivery of bioactive agents into cellular active sites by transferring the bioactive agent to natural LDL or other lipoproteins which may be more easily “taken up” into the cells of the patient, where the agent may produce a maximal affect. While not being limited by way of theory or mechanism, it is believed that transfer occurs from the microemulsions according to the present invention to the LDL or other natural lipoprotein via lipid transfer proteins such as cholesteryl ester transfer protein (CETP), phospholipid exchange protein and lipase. The term “lipid transfer protein” is used to describe a protein which facilitates the transfer of a lipid from the microemulsion according to the present invention to a natural lipoprotein or LDL.

Preferred embodiments of the lipoprotein according to the present invention utilize proteins with additional biological targeting capability which makes the system capable of targeted delivery of pharmaceutical agents to different biological sites in the body. For example, the use of avidin to attach biotinylated antibodies and other components as well as the use of lysozyme and hormones which may be used to attach to receptors at desired locations in the body, represents an additional approach to targeting of the present synthetic lipoproteins for delivery of drugs to patients.

The term “extended delivery” as used herein is understood to mean the release of therapeutic agents from microemulsions encapsulation over a period in excess of what would normally occur without the presence of stable microemulsions and generally in about 24 hours and in some embodiments as long as about 2 to 3 weeks.

“Structural integrity of microemulsions” as used herein shall mean the substantial maintenance of the pharmaceutical activity of the encapsulated substance during a period of extended delivery. This structural integrity is presumed to arise from the incorporation of lipidized polymer into the microemulsion formulations according to the present invention and the substantial maintenance of an entrapped bioactive agent, preferably a drug, for the period of extended delivery. Structural integrity may be imparted by adding lipidized polymer to the microemulsion in order to maintain the required microemulsion structure when challenged by the physiological conditions present in the subject animal. Alternatively, in some instances the inclusion of higher weight percentages of a steroidal component may also add to the structural integrity.

Compositions according to the present invention may be used to facilitate targeted therapy, especially targeted gene therapy. Lipidized polymers such as poly-L-lysine or polyethylenimine, among other cationic polymers, especially including polyamines, can be preparated in lipidized form and formulated such that the protein is associated with the surface of the microemulsion. These cationic polymers can be advantageously used for gene delivery since cationic polymers are known to complex with DNA through electrical attraction (interaction) between the negatively charged DNA and the positively charged polymers. (See attached FIG. 5).

While not being limited by way of theory, it is believed that the artificial LDL system of the present invention may also behave as native LDL by acquiring apolipoprotein E from the plasma. Embodiments of the present invention which incorporate apoliporoteins such as apolipoprotein B100 or apopliporotein E, in combination with lipidized protein represent further embodiments of the present invention.

Particular benefits of the compositions according to the present invention include the substantial enhancement of drug delivery to sites within the patient's body (targeting), the enhancement of drug entrapment upon formation of the microemulsions, the enhancement of the amount of drug per amount of lipid (loading), and the amount of hydrophobic drug which can be accommodated by this mode of delivery. Additional benefits of the present invention include the extended elaboration that microemulsion dosage forms according to the present invention exhibit when administered parenterally, especially via intramuscular, intramammary, intradermal, intraperitoneal, intra-ocular or subcutaneous routes. Topical dosage forms may also be formulated to exhibit extended release characteristics.

In a microemulsion-drug delivery system according to the present invention, the therapeutic agent preferably is encapsulated in the microemulsion (preferably, in the lipid core or amphipathic lipid monolayer or alternatively, at the surface of the microemulsion), associated with lipidized polymer and then administered to the subject being treated. Preferably, in certain embodiments, in order to take advantage of the extended elaboration characteristics of the dosage forms of the present invention, the dosage forms are administered parenterally via an intramuscular or subcutaneous route. The dosage forms of the present invention may also be administered via an intra-ocular, intramammary, intradermal or intraperitoneal route to produce a therapeutic dosage form having the characteristics of extended elaboration. In addition to the parenteral dosage forms, certain dosage forms of the present invention may be utilized in topical or oral formulations.

Compositions according to the present invention may be formed in the presence of solvent or an aqueous medium. As for the technique of preparation of the microemulsion, it is preferred to use a probe sonicator, such as the Branson 450 to form the microemulsion. Other homogenizing devices may also be used such as high shear mixers, high-pressure homogenizers, high shear impellers and the like. The compositions may be used immediately or stored prior to use.

Alternatively, the composition solutions may be dehydrated, thereby enabling storage for extended periods of time until use. Standard freeze-drying equipment or equivalent apparatus may be used to dehydrate the solutions containing the microemulsions. The solutions may also be dehydrated simply by placing them under reduced pressure. Alternatively, the microemulsions and their surrounding medium can be frozen in liquid nitrogen prior to dehydration. Dehydration with prior freezing may be performed in the presence of one or more protective sugars in the preparation including, for example, trehalose, maltose, sucrose, glucose, lactose and dextran, among others. When the dehydrated microemulsions solution is to be used, rehydration is accomplished by methods which include simply adding an aqueous solution, e.g., distilled water, to the microemulsions and allowing them to reformulate in solution.

In an alternative method embodiment, microemulsions comprising a bioactive agent according to the present invention are formulated and then incubated in the presence of a natural lipoprotein, preferably an LDL, and a lipid transfer protein, preferably cholesteryl ester transfer protein (CETP), phospholipid exchange protein (PEP) or lipase at a concentration and temperature (generally, within a range of approximately 30-40° C., preferably about 37° C.) under conditions which allow enzymatic reactions to take place in order to promote transfer of the bioactive agent in the microemulsion to the natural lipoprotein. In this method aspect according to the present invention, the resulting natural lipoprotein or LDL containing bioactive agent may be separated from the microemulsion and administered to the patient, or administered to the patient in combination with the microemulsion. A method which results in at least about 5% by weight of the bioactive agent in the microemulsion being transferred to the natural lipoprotein is preferred, at least 10% by weight of the bioactive agent in the microemulsion being transferred is more preferable and at least about 15-18% by weight of the bioactive agent being transferred is even more preferable.

The compositions according to the present invention are generally administered in association or in admixture with a pharmaceutically acceptable carrier selected with regard to the intended route of administration and standard pharmaceutical practice.

Bioactive agents for incorporation into microemulsions include, for example, those which have been described in the present specification as well as additional agents, not otherwise specified. The present compositions may accommodate a broad range of chemical characteristics ranging from hydrophobic (lipophilic) to hydrophilic (lipophobic) and are particularly appropriate for agents which are lipid soluble, but are difficult to deliver because of their hydrophobicity. Particular bioactive agents are understood to include analogues and derivatives of such agents including biologically active fragments unless otherwise indicated.

Pharmaceutical dosage forms of the present inventions may be comprised of microemulsions comprising a bioactive agent (preferably also including a lipidized protein associated therewith) and optionally, any suitable pharmaceutical carrier. A preferred class of carrier is aqueous including both distilled water and isotonic saline. Administration of high integrity microemulsions (i.e., those microemulsions associated with a lipidized protein) according to the present invention may be accomplished by any usual route with particular reference to the preferred routes of administration.

Preferred routes of parenteral administration as used herein include intracranially, intramuscular, intramammary, intraperitoneal, subcutaneous and intra-ocular administration. However, dosages adapted to parenteral administration may be used in a variety of administration methods, especially including topical and oral administration.

Having generally described the invention, reference is now made to the following specific examples which are intended to illustrate preferred and other embodiments and comparisons. The included examples are not to be construed as limiting the scope of this invention as is more broadly set forth above and in the appended claims.

### EXAMPLES

### Example 1

### Lipoprotein-Resembling Phospholipid-submicron Emulsion for Cholesterol-Based Drug Targeting, BCH

The objective of this experiment was to develop and evaluate lipoprotein-resembling phospholipid-submicron emulsion (PSME) as a carrier system for new cholesterol-based compounds for targeted delivery to cancer cells. BCH, a boronated cholesterol compound for boron neutron capture therapy (BNCT), was originally developed in our laboratory to mimic the cholesterol esters present in the LDL and to follow a similar pathway of cholesterol transport into the rapidly dividing cancer cells. The lipoprotein-resembling system was designed to solubilize and facilitate BCH delivery to cancer cells. BCH-containing PSME was prepared by sonication. Stock solutions of individual lipid and BCH were prepared in chloroform. Various lipids and BCH were mixed and the mixture was composed of the following ratio (w/w): Triolein: egg phosphatidylcholine: lysophosphatidylcholine: cholesterol oleate: cholesterol: BCH, 70: 22.7: 2.3: 3.0: 2.0: 2.0, respectively. All components were combined and chloroform was evaporated under a stream of nitrogen. The preparation was then desiccated overnight at 4° C. to remove residual solvent. Following addition of 10 ml of 2.4 M NaCl for 102 mg of lipid and BCH mixture, the preparation was sonicated under nitrogen for 30 min using a probe sonicator (Branson Sonifier 450) at output 5, while the temperature was maintained at 55° C. Inductively coupled plasma (ICP) and thin layer chromatography (TLC) were used to monitor possible degradation of BCH with the sonication condition.

The lipid structure of PSME and location of BCH in the formulation were assessed based on experimental results. Density gradient ultracentrifugation fractionated the emulsion into 3 particle size populations with structures and compositions resembling native lipoproteins. Chemical compositions and particle sizes of different PSME particles were determined. The following table shows the chemical composition:

TABLE 1
Composition of different fractions of BCH-containing PSME

| | | | | Cholesteryl | | |
|---|---|---|---|---|---|---|
| PSME | Particle | BCH | Cholesterol | oleate | Phospholipids | Triolein |
| Fraction | size | (% w/w) | (% w/w) | (% w/w) | (% w/w) | (% w/w) |
| First | 161 ± 2 | 2.4 ± 0.1 | 1.9 ± .1 | 3.0 ± 0.2 | 6.5 ± 0.6 | 86.3 ± 0.5 |
| Second | 76 ± 1 | 2.2 ± 0.1 | 2.3 ± 0.1 | 2.8 ± 0.1 | 12.6 ± 0.4 | 80.1 ± 0.2 |
| Third | 40 ± 3 | 1.8 ± 0.1 | 3.1 ± 0.2 | 2.5 ± 0.1 | 27.4 ± 0.4 | 65.3 ± 0.9 |
| Fourth | NA | 0.5 ± 0.1 | 3.7 ± 0.2 | 0.3 ± 0.1 | 89.3 ± 0.8 | 6.1 ± 0.1 |

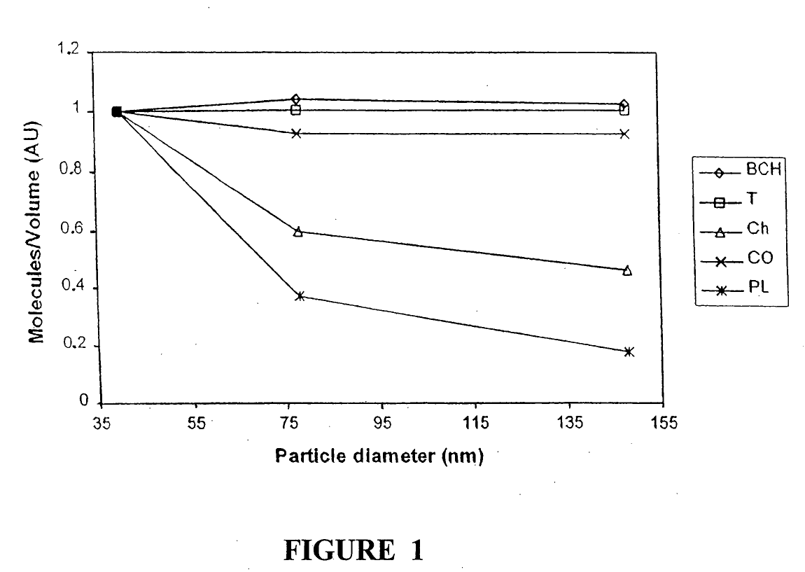

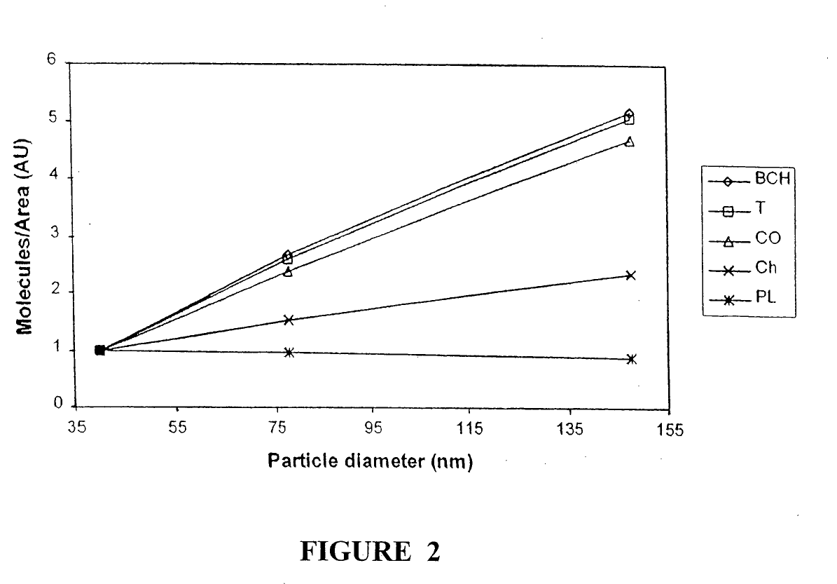

According to the chemical composition, the location of BCH, with respect to other lipids, was confirmed to be in the core of these lipoprotein particles the same as triolein and cholesteryl oleate, while phospholipids and cholesterol were present at the surface (See FIGS. 1 and 2). This indicates the composition and structure similarity between these submicron particles and native lipoproteins.

The Stability of the BCH-containing PSME formulation was also studied. Dialyzed BCH-containing PSME fractions were incubated at room temperature and frozen at 4° C. to study any possible hydrolysis of the drug (BCH) and the stability of these different particles. Samples were collected at day 1, 3, 10, and 38 for each fraction at different incubation temperatures and analyzed for BCH by HPLC. Particle size of each sample was also measured by photon correlation spectroscopy.

After 38 days the BCH and the formulation appeared to be unchanged. The particles size remained without any significant change during this period. HPLC analysis of the BCH showed that after 38 days at 4° C. about 85% of the initial BCH was recovered from the formulation.

Cell culture data showed sufficient uptake of BCH in rat 9L glioma cells (it is an important requirement to have about 20 μg of ¹⁰B per gram of cells to achieve successful BNCT) as shown in following table:

TABLE 2
BCH uptake in 9L glioma cell culture

| | | Concentration of B |
|---|---|---|
| | Initial concentration of | in the cells after 18 hrs |
| Experiment | B in the media (μg/ml) | incubation (μg/g cells) |
| Control | 0 | 0 |
| Low concentration ^{a} | 8.25 | 50 ± 10 |
| High concentration ^{a} | 16.5 | 61 ± 13 |

The lipoprotein-resembling PSME appears to be a novel carrier system that can incorporate a cholesterol-based compound, interact with native LDL and sufficiently deliver the compound into cancer cells in vitro. The sonication method used in the preparation didn't affect the compound or the lipids during the formulation. The formulation appears to be stable with minimal degradation after 38days. Our study suggests that these PSME particles have similar lipid composition and structural organization as native lipoproteins with the ability to interact and associate with LDL in vitro.

### Example 2

### BCH Distribution into Human LDL and Uptake by Human Glioma Cells 767SF

The purpose of this experiment was to study the efficiency of delivering newly synthesized boronated cholesterol, BCH, for boron neutron capture therapy to human glioma cells 767 SF. The drug was incorporated in a lipoprotein-resembling submicron emulsion to benefit from the increased uptake of the cholesterol in these cells due to the increased demand of the building new cell membrane for these rapidly dividing cancer cells. Also the similarity in structure between these BCH-containing submicron emulsion and native lipoproteins may contribute the dynamic exchange and transfer of lipid between different lipoproteins in the body and consequently the transfer of the boronated cholesterol to the cancer cells.

Methods: BCH-containing submicron emulsion was prepared by sonication at 55° C. of various lipids, the natural component of native lipoproteins, along with the boronated cholesterol. In vitro transfer of BCH to LDL was evaluated in LPDS and PBS. BCH uptake by human glioma cells 767 SF was compared when FBS or LPDS were used in the culture media. Results: After separating the submicron emulsion particles from the LDL, analysis of the LDL particles showed that about 18% of BCH originally incorporated in the second fraction of the submicron emulsion was transferred to the human LDL after 1 hr incubation at 37° C. in LPDS media. This transfer was not observed when the incubation was carried in PBS (no BCH was detected at the LDL fraction after incubation). This indicates that certain molecule in the serum, probably cholesteryl ester transfer protein CETP, was responsible for the interaction and transfer of BCH to the human LDL. This indicates also the unique resemblance between these submicron emulsion particles and serum lipoproteins as they exchange lipids in the presence of serum proteins. Lipid exchange between different classes of lipoproteins was established by others to be dependent on the presence of certain exchange proteins like CETP and phospholipid exchange protein. CETP was described as less selective to its substrate as it transfers not only cholesteryl esters (CE) but also triglycerides (Yokoyama et al, Magnes.Res., 7, (2) 87-105 (1994). This can be advantageous since drug molecules of similar structure of CE may also be a substrate for this enzyme and be transferred to body's lipoproteins when incorporated in this submicron emulsion. Drug transfer to LDL can be another attribute of this formulation to selective delivery via the LDL pathway.

To further investigate the use of this formulation in vitro we studied the cellular uptake of BCH in the presence and absence of lipoproteins, in FBS and LPDS. Results showed that uptake in FBS (lipoproteins are present) was more than triple the amount when the media contained no lipoproteins (LPDS) as shown in the following table:

BCH uptake in 767SF glioma cell culture

| | | Concentration of B |
|---|---|---|
| | Initial concentration | in the cells after 18 hrs |
| Experiment | of B in the media (μg) | incubation (μg/g cells) |
| Control | 0 | 0 |
| FBS ^{a} | 38 | 57.6 ± 15.5 |
| LPDS ^{a} | 38 | 14.7 ± 3.5 |

^{a} All values are means ± SEM of three experiments.
These results, in FBS, also meet the requirement for successful boron neutron therapy (>20 μg B/gm cells).

### Example 3

### Amphotericin B Incorporation in Lipoprotein-resembling Submicron Emulsion

Stock solutions of individual lipid were prepared in chloroform; Amphotericin B (AmpB) was dissolved in methanol. Various lipids and AmpB were mixed and the mixture was composed of the following in mg: Triolein: egg phosphatidylcholine: lysophosphatidylcholine: cholesterol oleate: cholesterol: AmpB, 70: 22.7: 2.3: 3.0: 2.0: 10, respectively. All components were combined and chloroform/methanol was evaporated under a stream of nitrogen. The preparation was then desiccated overnight at 4° C. to remove residual solvent. Following addition of 10 ml of 2.4 M NaCl for 110 mg of lipid and AmpB mixture, the preparation was sonicated under nitrogen for 30 min using a probe sonicator (Branson Sonifier 450) at output 5, while the temperature was maintained at 55° C. Particles were separated and dialyzed as described before. AmpB was analyzed by spectrophotometry after suitable dilution in methanol at 404.5 nm.

AmpB was successfully solubilized in the submicron emulsion system (2.33 mg of the added AmpB was incorporated in the system). Most of the incorporated AmpB (72%) was recovered with the first fraction of the submicron emulsion, which had particle size of 177 nm. AmpB is very effective antifungal drug; unfortunately it suffers from side effects when used parenterally (renal toxicity). Another limitation of this compound is its low water solubility. Incorporating AmpB in lipoprotein-resembling emulsion can lower its toxicity and solubilize the drug for successful parenteral administration.

### Example 4—Lipidized Lysozyme

As for the second component of the artificial LDL system, the lipidized protein- we have conducted our experiment on lysozyme as a model protein for the chemical lipidization. The following study describes the chemical reaction for the lipidization and its detection, which can be carried out for virtually any other protein having nucleophilic amine, hydroxyl or thiol (preferably amine) groups as well. The objective was to chemically modify lysozyme by the addition of fatty chains of stearic acid to the lysine amino group present in the protein and the detect the chemical modification by MALDI-TOF.

Methods

Preparation of Active Ester

3.45 g N hydroxysuccinamide was dissolved in dry ethyl acetate (150 ml). Then 8.53 g of stearic acid was added to that solution. A solution of dicyclohexylcarbodiimide (6.18 g) in dry ethyl acetate (10 ml) was added to the above reaction mixture and left overnight at room temperature. Dicyclohexylurea was removed by filtration and the filtrate yielded white crystal of the stearic acid active ester under rotavapor. The crystals were then recrystalized in ethanol,

Lipidization of Lysozyme

10 mg of the stearic acid active ester prepared above were dissolved in 1.5 ml of dimethylformamide (DMF) and then dropwise added while shaking to 2.5 ml solution containing 10 mg lysozyme in distilled water. The mixture was left overnight at 37° C. The mixture was then dried under reduced pressure and redissolved in distilled water and passed through a 0.45 syringe filter. A control reaction was also conducted under the same conditions but without the addition of stearic acid ester to eliminate any effect of the reaction conditions on the molecular weight of lysozyme.

MALDI-TOF Analysis

The modified lysozyme sample was analyzed by matrix assisted laser desorption ionization (MALDI) mass spectrometry using a Bruker Reflex time flight mass spectrometer (Billerica, Mass.) retrofitted with delayed extraction. The matrix was a saturated solution of 3.5 dimethoxy-4 hydroxycinamic acid (Aldrich, Milwaukee, Wis.) in a 50:50 mixture of water:acetonitrile with 0.1% triflouroacetic acid (TFA). The MALDI target was first spotted with nitrocellulose and allowed to dry. 2 (L of sample was applied next and dried. The sample was washed with cold water with 0.1% TFA. After washing two times, 0.5 (L of solvent was added to each sample. The spectrum was acquired in linear mode by averaging 26 laser shots and was externally calibrated using lysozyme MH+ and MH22+.

Results

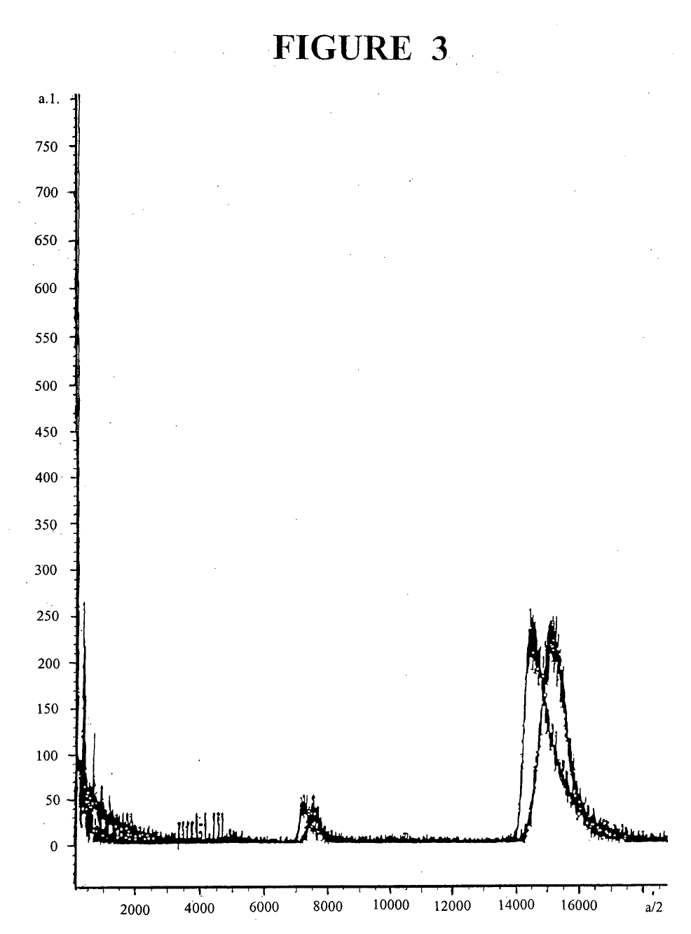

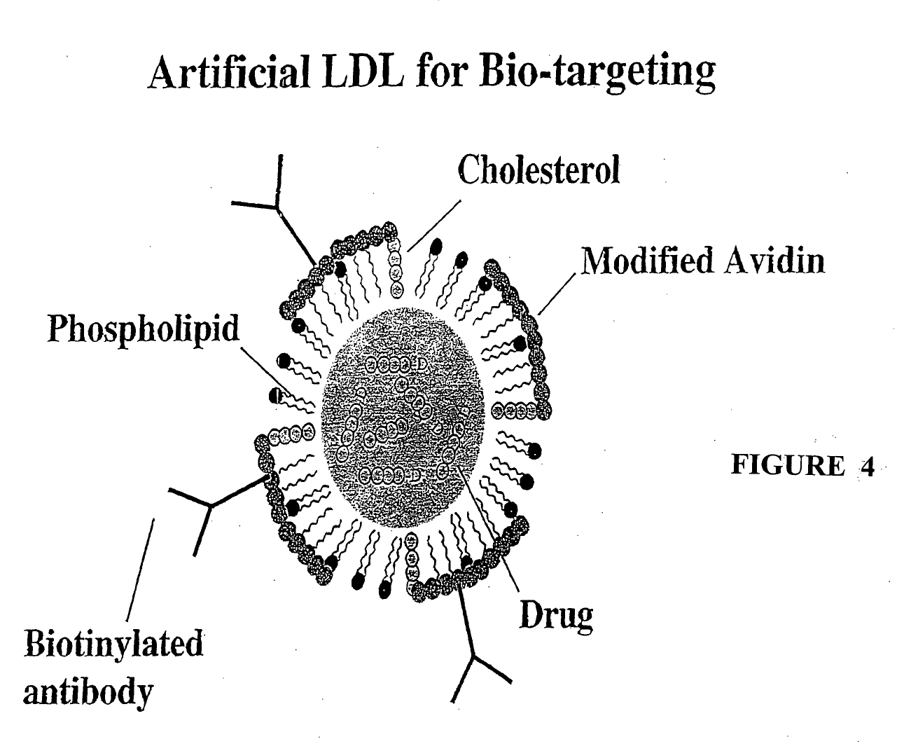

The mass spectrum of the modified lysozyme showed an obvious increase in the molecular weight of lysozyme (FIG. 3). The shifting range was around 1000 da, which represents the attachment of three fatty chains of stearic acid. There was no effect of the reaction media on the molecular weight of lysozyme when no active ester was added.

Conclusions

Chemical lipidization of lysozyme was achieved. MALDI-TOF is an appropriate tool to evaluate the extent of reaction in producing lipidized protein. This method may be used readily to modify other proteins in a similar way.

Since the chemical lipidization is an important element to promote the attachment of proteins to the microemulsion system according to the present invention, one can readily lipidize virtually any protein in a similar manner. Other polymers may be modified in similar manner and used to target different tissues in the body.

### Example 5

### Lipidized Polylysine and its Association with Lipoprotein-resembling Submicron Emulsion for Gene Delivery

This experiment describes the lipidization of positively charged polymer, polylysine, and its association with the lipoprotein-resembling submicron emulsion particles. Polylysine has been used to condense DNA through charge interaction. Cationic lipids in liposome formulation have been also used to carry DNA using the same concept of charge interaction. Unfortunately, cationic lipids are very toxic and their use is limited for in vitro transfection of DNA. In this patent we describe a system, which utilize the positive charge of cationic polymers and the biocompatible formulation of lipoprotein resembling submicron emulsion system to deliver negatively charged DNA.

Methods:

Preparation of Lipoprotein-resembling Phospholipid Submicron Emulsion

Stock solutions of individual lipid were prepared in chloroform. Various lipids were mixed and the mixture was composed of the following ratio (w/w): Triolein: egg phosphatidylcholine: lysophosphatidylcholine: cholesterol oleate: cholesterol, 70: 22.7: 2.3: 3.0: 2.0, respectively (reference maranhoa and van Berkel). All components were combined and chloroform was evaporated under a stream of nitrogen. The preparation was then desiccated overnight at 4° C. to remove residual solvent. Following addition of 10 ml of 2.4 M NaCl for 102 mg of lipid and BCH mixture, the preparation was sonicated under nitrogen for 30 min using a probe sonicator (Branson Sonifier 450) at output 5, while the temperature was maintained at 55° C.

Lipidization of Poly-_{L}-lysine

N-alkylation of poly lysine (PLL) was achieved as described by Kim et al., J Controlled Release, 47, pp. 51-59 (1997), with modification. In brief, 30 mg PLL was dissolved in 2 ml DMSO. 10 μl of triethylamine was added to the mixture. Palmitoyl chloride (20 mg) was used to react with the ε-amino of the lysine in the poly lysine polymer. The mixture was allowed to react at room temperature for 2 hrs. The mixture was filtered and acetone was added to the filtrate to precipitate the lipidized polymer, palmitoyl poly lysine. The product was dissolved in methanol, reprecipitated by acetone, and dried under vacuum overnight. The modified polymer was characterized by proton NMR.

Interaction of Lipidized Poly Lysine with Lipoprotein-resembling Submicron Emulsion

The fractions of different sizes of the phospholipid submicron emulsion were incubated individually (TEFF 50 μl of each fraction or 20 μl LDL+30 μl PBS) with 100 μg or 50 μg of polylysine or palmitoyl polylysine for 1 hr in 2 ml PBS at 37° C. with gentle shaking. Agarose gel electrophoresis was performed according to the method described by Greenspan et al., Electrophoresis, 14, pp. 65-68 (1993) using Nile Red as the fluorescent dye to determine the electrophoretic mobility of the lipoprotein-resembling submicron emulsion and to examine its interactions with lipidized and native poly lysine. In brief, 0.6% agarose gel was prepared in 50 mM barbital buffer, pH 8.6. Five μl of Nile Red in acetone (100 μg/ml) was dried out in test tube. The incubation sample was then added to the tube individually (50 μl sample to each tube) and mixed until Nile Red was in solution. Five μl of sucrose solution (30%, w/v) was added. Each electrophoretic well was loaded with 11 μl of sample preparation. Electrophoresis was conducted for 1 hr at 56 V at room temperature. Different electrophoretic bands on the gel were visualized under UV lamp.

Degree of Lipidizedpolylysine Association with PSME

In order to measure the amount of lipidized PLL associated with the submicron emulsion particles, (250 μl of 2nd fraction blank containing 2.391 mg T/ml) of the second fraction were incubated with 2 mg of lipidized poly lysine in 2 ml PBS for 1 hr at 37° C. with gentle shaking. The density of the mixture was adjusted to 1.08 g/ml with solid KBr and placed in the bottom of 13.5 ml centrifuge tubes; the remaining of the tube was filled with KBr solution of 1.063 g/ml. The mixture was then subjected to density gradient ultracentrifugation at 285,000 g for 2 hrs, and centrifugation was allowed to stop without use of break. The top 4 ml, where the emulsion particles are recovered, and the bottom 5 ml of the tubes were collected and assayed for content of lipidized polylysine using the modified Lowry method. Tubes containing lipidized PLL only, and submicron emulsion only were centrifuged along with the mixture samples and used as controls.

### Results

Particle size and chemical composition

| | Particle | Cho- | Cholesteryl | | Phospho- |
|---|---|---|---|---|---|
| Fraction | size (nm) | lesterol % | oleate % | Triolein % | lipid % |
| F1 | 155 | 1.910076 | 6.448696 | 84.34123 | 7.3 |
| F2 | 76 | 2.123761 | 6.202347 | 79.07389 | 12.6 |
| F3 | 44 | 2.706742 | 5.079775 | 64.81348 | 27.4 |

Degree of Lipidized Poly Lysine Association with PSME

The amount of 545 μg of m-PLL per 1 mg of triolein associated with the 2^{nd} emulsion fraction. This amount was calculated after subtracting the amount of m-PLL from control tube of m-PLL alone that floats to the top and the interference of emulsion turbidity on the analysis of m-PLL associated with the emulsion particles.

Interaction of Lipidized Poly Lysine with Lipoprotein-resembling Submicron Emulsion

Due to the association of m-PLL with submicron emulsion particles the surface charge of the particles was completely reversed. Before addition of m-PLL the emulsion particles had slight negative charge as the case with human LDL too. After the addition of m-PLL which has a positive charge, owing to the free ε-amino groups of lysine, the particles showed movement toward the negative electrode suggesting the charge of the m-PLL associated submicron emulsion is positive. The addition of unmodified polylysine caused the emulsion particles to precipitate immediately. The precipitation can be due to charge neutralization rather than physical incorporation of the polylysine molecules with the surface of the emulsion particles. Lipidization of polylysine appears to be critical for successful association with the emulsion without precipitation. Since DNA is negatively charged, the m-PLL associated emulsion particles has the ability to carry DNA molecules, which can be used for gene delivery. Experiments comparing emulsion compositions without the lipidized polylysine (did not carry DNA molecules) and those with lipidized polylysine (efficiently carried the DNA molecules) confirm these results.

### Example 6—Lipidized Avidin

Avidin is a glycoprotein which can be lipidized readily using the above-described methods and associated with the microemulsion system of the present invention, which can then function as a universal station for the attachment of various biotinylated proteins (for example, enzymes, hormones, toxins, antibodies, receptors, etc.) polymers and/or bioactive agents, such as drugs, including those biotinylated proteins which are well known in the art. Lipidized avidin can also be associated with native LDL and therefore allow the binding of biotinylated polymers, protein and drugs to benefit from the LDL pathway for delivery.

The advantage of using this system with a biotinylated antibody include the intact functionality of the antibody (FIG. 4). The antibody will be functional since the active site has not been modified or affected. The approach will also overcome any possible limitations of non-selective modification and disorientation of the antibody in the microemulsion which might occur from direct modification on the antibody

The advantage of using this system with polymers includes the facilitation for targeted gene delivery. Polymers can be lipidized in a similar manner as proteins. A cationic polymer (e.g, polylysine or polyethyleneimine) in a lipidized form which is associated with the microemulsion can be used for gene delivery since cationic polymers are known to complex with DNA through electrical attraction between the negatively charged DNA and positively charged polymers (FIG. 5).

This artificial LDL system may also behave as native LDL by acquiring apolipoprotein E from the plasma. Apolipoprotein E is known to interact with LDL receptors in a similar manner to Apolipoprotein B 100 but with even higher affinity. Apolipoprotein B 100 is the main protein component present in the native LDL and responsible for interaction with the LDL receptor. Interaction with an LDL receptor initiates endocytosis for the entire LDL particle and therefore the process can be utilized to deliver drug molecules inside the cells.

The following examples illustrate the synthesis of carborane cholesterol compounds (BCH) which may be used in the present invention. The synthesis of the carborane cholesterol compounds according to the present invention is presented in the following experiments. In general, where solvent is used, it is dried and distilled prior to use. Nitrogen is used dry at all times. All other materials are dried and distilled prior to use.

### Experiment 7

### Synthesis of Carborane acid (1-hydroxycarbonyl-1, 12-dicarba-closo-dodecaborane)

n-BuLi (1.1 ml, 1.66 mmol, 1.6 M in hexane) was slowly added to a stirred solution of p-carborane (200 mg, 1.38 mmol) in ether (80 ml) in a flask fitted with a reflux condenser at 0° C. The reaction mixture was warmed to room temperature and refluxed for 3 hr. The reflux condenser was removed and the reaction mixture was cooled to −78° C. (dry ice/aceton). Dry ice (CO₂) was added to the reaction mixture under positive flow of nitrogen. The reaction mixture was allowed to warm to room temperature and excess ether was removed by vacuum. The residue was dissolved in 10 ml water and extracted with ether (2×5 ml). The aqueous layer was acidified with HCl solution (5M) to pH 1. The product, p-carborane carboxylic acid, was extracted by ethyl acetate. Proton NMR and ¹³C NMR have been used to confirm the structure of product. ¹H NMR (CDCl₃, 400 Hz) δ: 1.6-3.2(10H, B-H)(see FIG. 2); ¹³C NMR (400 Hz, DMSO-d₆) δ: 166.58, 83.33;

### Experiment 8

### Synthesis of cholesteryl 1,12-dicarba-closo-dodecaborane 1-carboxylate

p-carborane carboxylic acid of 80 mg was placed in 25 ml flask. Thionyl chloride (5 ml) was added and the flask was quickly attached to reflux condenser protected by drying tube. The assembly was mounted in oil bath at 78° C. for 4 hr. Excess SO₂Cl was removed by vacuum. Cholesterol (160 mg) was dissolved in methylene chloride (3 ml) (containing pyridine 50 μl). The reaction mixture was stirred under nitrogen for 48 hr. The excess solvent was removed and residue was subject to column chromatography to separate the product from impurity. Proton NMR and ¹³C NMR have been used to confirm the structure of product. ¹H NMR (CDCl₃, 400 Hz) δ: 1.6-5.1(10H, B-H), 5.34(1H, Chol 6), 4.43(1H,Chol 3); ¹³C NMR (CDCl₃) δ: 61.72(acid 1), 139.24 (chol 5), 122.71 (Chol 6);

### Experiment 9

### Synthesis of cholesteryl 1,12-dicarba-closo-dodecaborane 1-carboxylate

A mixture containing p-carborane carboxylic acid (80 mg, 0.4 mmol), cholesterol (180 mg, 0.4 mmol), DCC (dicyclohexylcarbodiimide) (80 mg, 0.4 mmol), DMAP (4-(dimethylamino) pyridine) (10 mg, 0.08 mmol) was stirred in 10 ml methylene chloride for 48 hr at room temperature. The reaction mixture was cooled to 0° C. and precipitate was filtrated. The filtrate was subject to silica gel column chromatography to purify the product: cholesteryl 1,12-dicarba-closo-dodecaborane 1-carboxylate. Proton NMR and ¹³C NMR have been used to confirm the structure of product. ¹H NMR (CDCl₃, 400 Hz) δ: 1.6-5.1(10H, B-H), 5.34(1H, Chol 6), 4.43(1H, Chol 3); ¹³C NMR (400 Hz, CDCl₃) δ: 61.72 (acid 1), 139.24 (chol 5), 122.71(Chol 6);

Each of the o-carborane cholesterol and m-carborane cholesterol compounds may be synthesized by analogy to the p-carborane cholesterol compound synthesized above in examples 7-9 (example 9 is BCH) substituting o-carborane or m-carborane for p-carborane in the syntheses. The remaining steps would proceed as they are presented, above. In sum the following three compounds are presented: cholesterol 1,12-dicarba-closo-dodecaborane 1-carboxylate, cholesterol 1,2-dicarba-closo-dodecaborane 1-carboxylate, and cholesterol 1,7-dicarba-closo- dodecaborane 1-carboxylate.

DNA Delivery

In the following set of examples, the embodiment directed to the delivery of nucleic acids, in particular, DNA to cells, in a more particular embodiment as part of a cancer treatment modality, and more particularly as part of gene transfection for the treatment of cancer or other disease states. Gene transfection can be defined as the delivery to and subsequent expression of functional genetic material in specific cells to manipulate their intrinsic genetic profiles. During the last decade, researchers involving gene transfection have been expanding very rapidly and many gene delivery systems have been developed to efficiently transfect various cells in both in vitro and in vivo experimental conditions. As an effective gene delivery system, it must be able to carry sufficient amount of genetic material and express the genetic information in specific cells resulting in a significant changes in genetic profiles. In general, genetic materials can be carried and expressed in specific cells by either viral vector systems or non-viral vector systems. The viral vector systems, including retrovirus, adenovirus, adeno-associated virus, herpes simplex virus and lentivirus, have been extensively investigated owing to their high transfection efficiency. However, their applications are limited by their complicated handling procedures for in vitro experiments and poor safety profiles for in vivo studies (1-2). Compared to the viral vector systems, the non-viral vector systems are easy to handle and have better safety profiles. Consequently, the development of effective non-viral gene delivery systems has become the center pieces in many research laboratories (3-5).

Since lipids are the main components of cell membrane, most non-viral vectors are lipid-based such that the vectors can be effectively incorporated into cell membrane and facilitate the delivery of genetic materials into specific cells. Among these non-viral vectors, cationic liposomes, which carry positive charge and electrostatically interact with negatively charged DNA to form complexes, are most widely studied (6-11). However, the success of using cationic liposomes for gene transfection is partly hampered by the cytotoxicity of the cafionic lipids. Polymer-based non-viral vectors have also been widely investigated, including poly-L-lysine, polyethenimine, polyamidoamine dendrimer, and chitosan (12-20). One main disadvantage of these systems is the low efficiency of transfection. Recently, Kim et al. developed a new gene delivery system called Terplex system, which is based on a complex formed by natural low-density lipoprotein (LDL) and stearyl-poly-L-lysine (21-22). Through hydrophobic interaction, stearyl-poly-L-lysine can be incorporated into the LDL particles. The assembled complex possesses positive charge and was able to carry negatively charged DNA and successfully deliver the DNA into vascular smooth muscle cells.

This application is directed inpart to the development and evaluation of a novel artificial lipoprotein delivery system that can carry DNA materials for effective in vitro gene transfection in tumor cells. Similar to the structure of natural lipoproteins, this artificial lipoprotein delivery system consists of nanoemulsion cores made of natural lipids and surface lipidized poly-L-lysine, which replaces the surface protein as in natural lipoproteins. With proper weight ratio of poly-L-lysine to the lipids in nanoemulsion, the artificial lipoprotein delivery system efficiently carried plasmid DNA containing β-galactosidase gene and transfected human SF-767 glioma tumor cells. Our experiments showed that because the lipids used in the system are all natural substances, the cytotoxicity of this delivery system could be significant lower than the commercial gene transfection systems using cationic liposomes. The benefit associated to the low cytotoxicity makes it especially useful as an alternative to Lipofectamine™ or other commercial gene transfection systems. Another advantage of this system is that it can be readily assembled using commercial available materials including phospholipids, cholesterol and poly-L-lysine. The chemical composition, particle size and type of surface poly-peptide or surface protein can be controlled and optimized allowing widely-diversified gene or drug delivery applications.

### Materials and Methods

Materials

Triolein (99%), egg yolk phosphatidylcholine (99%), cholesterol (99%), poly-L-lysine hydrobromide (MW 57900 Dalton based on viscosity), chloroquine (99%), o-nitrophenyl-β-D-galactopyranoside (ONPG), and 3-(4,5-dimethylthiazol-2-yl)-2,5-diphenyl tetrazolium bromide (MTT) were purchased from Sigma (St. Louis, Mo., USA). L-α-lysophosphatidylcholine (99%) was purchased from Avanti (Alabaster, Ala., USA). Cholesterol oleate (99%) was obtained from Acros (Pittsburgh, Pa.). 5-bromo-4-chloro-3-indoyl-β-D-galactoside (X-Gal) was from Life Technologies (Rockville, Md., USA). Electrophoretic grade agrose was purchased from FMC Bioproducts (Rockland, Me., USA). All other chemicals were of analytical grade obtained from Sigma or J. T. Baker (Phillipsburg, N.J., USA).

Preparation of Nanoemulsion

The oil phase of emulsion was composed of triolein (70%), egg phosphatidylcholine (22.7%), lysophosphatidylcholine (2.3%), cholesterol oleate (3.0%) and cholesterol (2.0%). The lipid components were dissolved in chloroform individually and then mixed thoroughly. The chloroform was then removed completely by a stream of nitrogen gas. In each 100 mg of lipid mixture, 10 ml of 2.4 M NaCl solution was added. The mixture was sonicated under nitrogen flow for 30 min using Model 450 Sonifier® (Branson Ultrasonics Corporation, Danbury, Conn.) with a duty cycle dial setting of 90% at output of 40 watts. The temperature of the mixture was maintained at 55° C. during sonication. The prepared emulsion was then passed through an Emulsiflex B3 device (Avestin, Ontario, Canada) at a pressure of 70 psi for 10 times to reduce the particle size to nanometer level. The emulsion was dialyzed against phosphate-buffered saline (PBS) using Spectra/Por® 2 molecularporous membrane tubing with molecular weight cut-off of 6000-8000 dalton (Spectrum Medical Industries, Inc. Houston, Tex.). The emulsion particle size distribution was measured by Submicron Particle Sizer Autodiluter Model 370 (NICOM Particle Sizing System, San Barbara, Calif.). In addition, the nanoemulsion was stored at room temperature and the particle size distribution was measured in 2, 4, 8, 16 weeks, respectively, to examine the stability of the nanoemulsion particles.

Lipidization of Poly-L-lysine

Lipidization of poly-L-lysine was performed as described by Kim et al. (21) with slight modification. Briefly, poly-L-lysine hydrobromide (30 mg) was dissolved in 2 ml DMSO in a 50 ml round-bottom flask. After triethylamine (10 μl) was added, palmitoyl chloride (20 mg) was added to the mixture to react with the amino group of the lysine residues in poly-L-lysine. The mixture was allowed to react at room temperature for 2 hrs and filtered. Acetone was added to the filtrate to precipitate the lipidized polymer, palmitoyl poly-L-lysine or abbreviated as p-PLL. The product was dissolved in methanol, re-precipitated by acetone, and dried under vacuum overnight. The modified polymer was characterized by proton NMR.

Incorporation of p-PLL into Nanoemulsion Particles

In each 1.5 ml microcentrifuge tube, nanoemulsion (50 μl) was diluted with 0.2 ml PBS solution and incubated with various amount of p-PLL at 37° C. based on the weight ratio of p-PLL to triolein in nanoemulsion. The weight ratios of p-PLL to triolein in the mixture were 0.125:1, 0.25:1, 0.5:1, and 1:1, respectively. After incubation for 1 hour, the mobility of the nanoemulsion particles in electric field was examined by agarose gel electrophoresis using Nile Red as the fluorescent dye. Agarose gel (0.4%) was prepared in TAE buffer (40 mM Tris-acetic acid, 1 mM EDTA, pH 8.0). Five μl of Nile Red solution in acetone (100 μg/ml) was dried out in test tube and redissolved in 30 μl of the incubation mixture as described above. In each sample, 6 μl of glycerine was added to increase the density of the sample and the sample (30 μl) was loaded in each sample well of the agarose gel. Electrophoresis was conducted for 1 hr at 70 volts at room temperature using Horizontal Mini-gel System (CBS Scientific Company Inc. Del Mar, Calif., USA). The mobility of the particles in electric field was visualized by Eagle Eye II Video System (Stratagene, Calif., USA).

Amplification and Purification of Plasmid DNA

Plasmid DNA, pSV-P-Galactosidase Control Vector, was purchased from Promega (Madison, Wis.) and was introduced into Epicurian Coli® XL1-Blue MRF′ (Stratagene, Calif.) by using standard transformation protocol. The transformed E. coli strain was maintained in Luria-Bertani (LB) medium containing 15% of glycerol at −80° C. To amplify the plasmid DNA, the E. coli strain was cultured in LB medium containing 100 unit/ml of ampicillin at 37° C. overnight and the cells were harvested by centrifugation. Plasmid DNA in the cells was extracted and purified using Wizard Plus SV Minipreps DNA Purification System (Promega, Madison, Wis.). The purity of plasmid DNA was confirmed by determining the ratio of optical absorbance at 260 nm and 280 nm (>=1.8) and further by 0.6% agarose gel electrophoresis. The agarose gel was stained with ethidium bromide (0.5 μg/ml) for 15 minutes and destained with deionized water for 10 minutes. DNA bands in the agarose gel were visualized by Eagle Eye II Video System. The concentration of plasmid DNA was determined by spectrophotometer at wavelength of 260 nm (1 OD₂₆₀≈50 μg/ml).

Assembly of the Complex of Nanoemulsion, p-PLL and Plasmid DNA

Nanoemulsion (50 μg) in 0.2 ml PBS was mixed with various amount of p-PLL in the same way as described above. The weight ratios of p-PLL to triolein in the nanoemulsion were 0.0625:1, 0.125:1, 0.25:1, 0.5:1 and 1:1, respectively. After incubation at 37° C. for 1 hour, DNA (2 μg) was added and incubated at room temperature for 15 minutes. Samples were then loaded into 0.4% agarose gel and the electrophoresis was performed as described above. Zeta potential and mobility of the assembled particles are measured by Submicron Particle Size Analyzer 90Plus (Brookhaven Instrument Corporation, Holtsville, N.Y., USA). Before they were measured for zeta potential, the samples were diluted with sodium nitrate solution (1 mM, pH 7.4) until the count of particles in the sample reached 100-300 kilo-counts per second (KCPS). Water and solutions used in zeta potential measurement were filtered with 0.1 μm Supor Acrodisc (Gelman Sciences, Ann Arbor, Mich., USA). The particle size, zeta potential and mobility was recorded by the built-in PC computer system.

Gene Transfection Experiment

Human glioma cell line SF-767 was obtained from the tissue bank of Brain Tumor Research Center (University of California-San Francisco, San Francisco, Calif., USA) and used in our transfection experiment because of its characteristics of aggressive growth. The cells were grown at 37° C. in 5% CO₂ with Eagle's Minimal Essential Medium (EMEM) medium supplemented with 10% fetal bovine serum (BioCell Laboratories, Rancho Dominguez, Calif., USA), 100 units/ml penicillin, and 100 μg/ml streptomycin. Culture was passaged twice a week to maintain the cells in exponential growth. The transfection was conducted in 6-well (35-mm in diameter) culture plates. SF-767 cells were seeded with 3×10⁵ cells in each well 24 hours before the transfection. During the day of transfection, nanoemulsion (50 μl) in 0.2 ml PBS was mixed with p-PLL in the ratios as described above, and incubated at 37° C. After 1 hour of incubation, 2 μg of plasmid DNA was added and incubated at room temperature for 15 minutes to obtain the complex of nanoemulsion/p-PLL/DNA. Cells were washed with PBS buffer for three times and 1 ml of EMEM (without serum and antibiotics) was added in each well. The nanoemulsion/p-PLL/DNA complex was added to each well and mixed with the medium completely by swirling. The cells were then incubated at 37° C. in 5% CO₂ for 12 hours before 1 ml EMEM medium containing 20% fetal bovine serum was supplemented. The cells were incubated for additional 24 hours. Both the nanoemulsion/p-PLL complex and the naked DNA were used, respectively, as the negative controls. As the positive control, Lipofectamine™ reagent purchased from Invitrogen (Carlsbad, Calif., USA) was incubated with the DNA and the transfection experiment was performed at the same condition.

Detection of β-galactosidase by X-Gal Staining and Enzymatic Assay

After 24 hours of transfection incubation, the cells in each well were washed twice with PBS buffer and then fixed with 2 ml fixing solution (2% formaldehyde and 0.2% glutarldehyde in PBS buffer) for 15 minutes at room temperature. After the cells were washed for three times with PBS, 1.5 ml of staining solution (5 mM potassium ferricyanide, 5 mM potassium ferrocyanide, 1 mM MgSO₄, 1 mg/ml X-Gal from 20 mg/ml stock in dimethyl formamide) was added. The cells were incubated at 37° C. in 5% CO₂ for 30 hours before the transfection was evaluated by light microscope. β-Galactosidase activity of cells was determined by β-Galactosidase Enzyme Assay System (Promega, Madison, Wis., USA). In brief, the cells from each well of the plate were trypsinized and collected by centrifugation.

They were disrupted by mixing with 200 μl of Lysis Buffer and incubating for 30 minutes at room temperature. The protein concentration of the cell lysate was determined by Bradford method (23). Cell lysate (100 μl) was mixed with 100 μl of ONPG solution in 2× Assay Buffer (1.33 mg/ml) and incubated in water bath at 37° C. for 5 hours. The enzymatic reaction was terminated by adding 300 μl of 1 M sodium carbonate solution. After the reaction mixture was diluted, the absorbance at 420 nm was read in a spectrophotometer (Baush & Lomb Spectronic 2000, Rochester, N.Y., USA). The enzymatic activity unit was defined in a similar way as described by Kim et al. (21).

Effect of Chloroquine on the Transfection Efficiency

In order to demonstrate the effect of chloroquine, which is a lysomotropic agent, on the transfection efficiency, chloroquine solution in PBS was added to the cell culture (80% confluent) with a final concentration of 100 μM. After 30 minutes of incubation at 37° C. and 5% CO₂, the culture medium was removed and the cells were washed with PBS for three times. Fresh medium was supplemented before the transfection experiment was started. The complex of nanoemulsion (50 μl in 0.2 ml PBS) with p-PLL (p-PLL:triolein=0.25:1) was used as the carrier for 2 μg of plasmid DNA in the transfection experiment. After transfection incubation, the cells were trypsinized and collected by centrifugation. The β-galactosidase activity was measured by the method as described above.

Cytotoxicity Comparison of Nanoemulsion/p-PLL Complex and Lipofectamine

Cellular toxicity of the nanoemulsion system was tested according to the MTT method reported by Mosmann (24). A complex of nanoemulsion (50 μl in 0.2 ml PBS) with p-PLL (p-PLL:triolein=0.25:1) was prepared in similar way as described above. In 96-well microplate, SF-767 cells were seeded at 2×10⁴ cells in each well containing 0.1 ml of EMEM medium. After 24 hours of incubation at 37° C. and in 5% CO₂, the nanoemulsion/p-PLL complex or Lipofectamine reagent was added to the cell culture. The amount of nanoemulsion/p-PLL complex or Lipofectamine reagent added to the cell culture was determined such that similar transfection efficiency could be obtained based on the transfection experiments. The cell culture grown on EMEM medium without nanoemulsion/p-PLL complex or Lipofectamine reagent was used as the control. Since only living cells are able to cleave the tetrazolium ring to produce dark blue crystals, which can be measured calorimetrically, the viability of cells after additional 1, 2, 3, and 4 days of growth was determined by measuring the ability of the cells to degrade tetrazolium salt MTT. Briefly, 25 μl of MTT solution in PBS buffer (0.5 mg/ml) was added to each well of culture and incubated at the same condition for additional 4 hours. The medium was then removed and 150 μl of DMSO was added to each well and mixed thoroughly until all the dark blue crystals were dissolved. The plate was read on an OPTImax Tunable Microplate Reader (Molecular Devices Corporation, Sunnyvale, Calif., USA) at wavelength of 550 nm. The cell viability was calculated and expressed as (ODₜᵣₜ/OD_{ctrl})×100%, where ODₜᵣₜ is the optical absorbance from the culture treated with either nanoemulsion/p-PLL complex or Lipofectamine reagent, and OD_{ctrl} is the optical absorbance from the culture of control.

### Results

Preparation of the Nanoemulsion and its Size Distribution

With the proper control of temperature (55° C.) during sonication, the obtained emulsion appeared to be homogeneous. The number weighted mean size of the emulsion particle was 110.2±42.9 nm. Since the emulsification was conduced by metal probe sonication, chelating agent (0.1 mM EDTA) was added to remove the free iron ion before it is dialyzed against PBS overnight (PBS changed every 6 hours). Followed by 10 cycles of size reduction by Emusiflex B3 device (Avestin, Ottawa, Canada), the number weighted mean particle size was reduced to 48.9±19.8 nm.

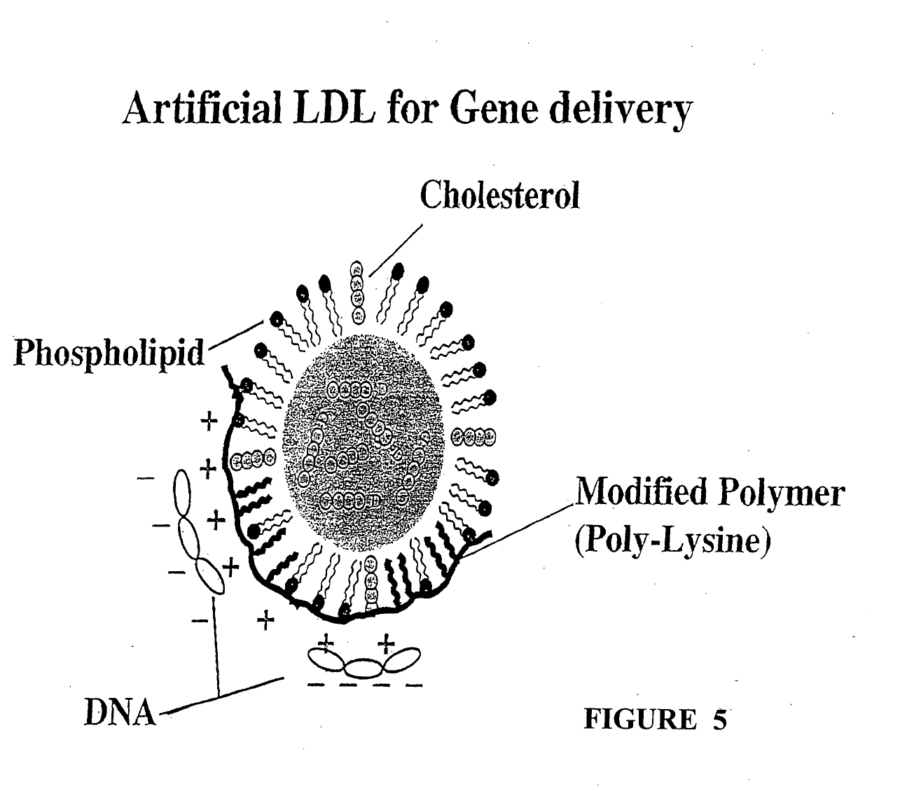

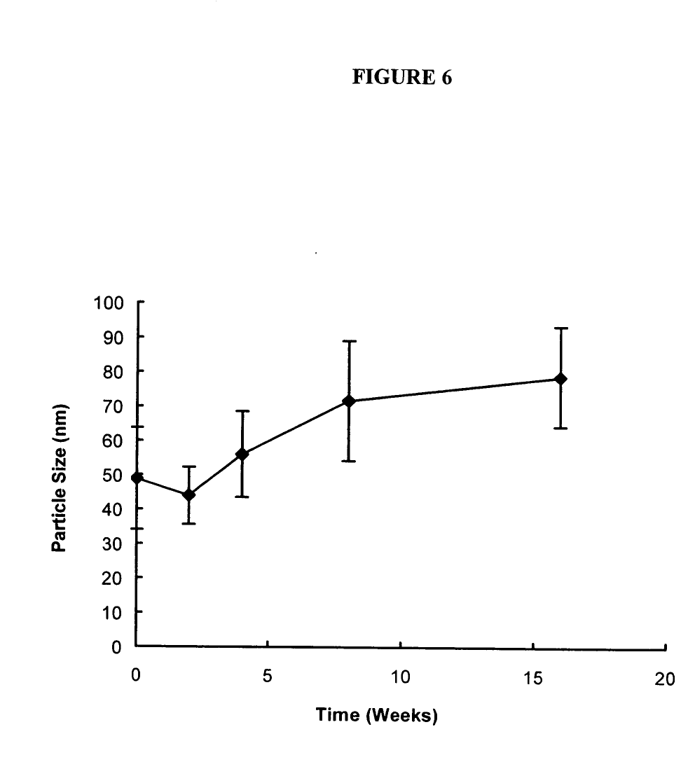

The physical stability of the nanoemulsion can be investigated by measuring the change in size distribution of the particles. The size distribution of the nanoemulsion particles was measured at 0, 2, 4, 8, 16 weeks after the preparation and results are shown in FIG. 6. The size distribution did not change significantly upon storage at room temperature. After 16 weeks of storage at room temperature, the size of the emulsion particles was 78.9×14.6 nm, indicating that the nanoemulsion particles are rather stable.

Incorporation of p-PLL into Nanoemulsion Particles

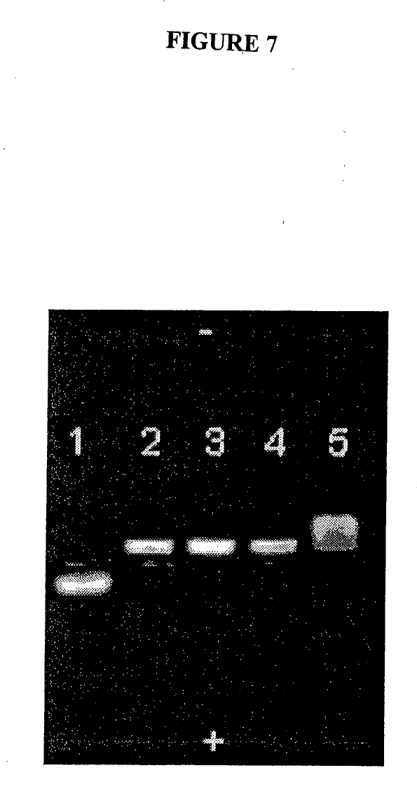

Since p-PLL is positively charged, the incorporation of p-PLL molecules into nanoemulsion particles will result in a change in surface charge of the particles. The change can be clearly seen in the picture of agarose electrophoresis (FIG. 7). Nanoemulsion particles moved to anode since they were negatively charged (Lane 1). The negative surface charge of the nanoemulsion particles was also confirmed by the zeta potential and mobility, which was −42.28±2.3 mV and −3±0.06 (m/s)/(V/cm), respectively. The incorporation of p-PLL neutralized the surface charge (Lane 2 to Lane 4) and resulted in the retardation of move in the electric field. When they were incubated with sufficient amount of p-PLL, the surface charge of particles was reversed to be positive and moved towards opposite direction in the electric field (Lane 5). The results indicated that p-PLL could be incorporated into the nanoemulsion particles.

Interaction of p-PLL Associated Nanoemulsion with DNA

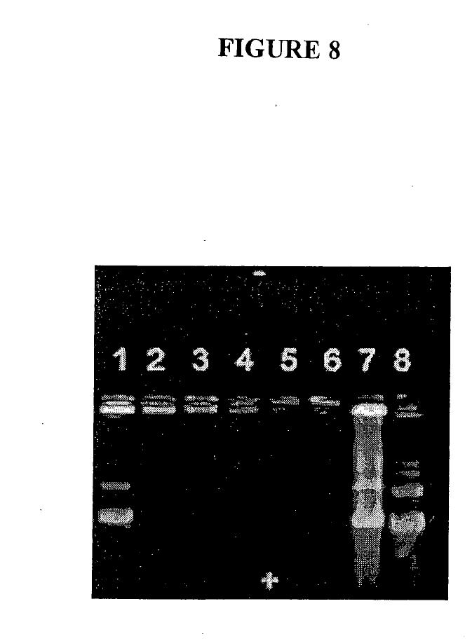

After incorporation of sufficient amount of p-PLL molecules into nanoemulsion particles, the complex carried a positive charge and can electrostatically interact with negatively charged DNA molecules. As indicated in FIG. 8, plasmid DNA (Lane 1) migrated towards the positive anode. When DNA plasmid was incubated with p-PLL (Lane 2), no DNA migration was observed, possibly because DNA molecules were bound by p-PLL and thus the ethidium bromide molecules could not intercalate into the DNA molecules resulting in no fluorescence emission. Lane 3 to Lane 7 showed the change in DNA carrying capability of the complex resulted from different ratios of p-PLL to nanoemulsion (i.e. the p-PLL to triolein ratio). At a high ratio of p-PLL to nanoemulsion, DNA was tightly held by the complex and thus no DNA migration band appeared (Lane 3 to Lane 6). When the ratio of p-PLL to nanoemulsion became sufficiently low (0.0625:1 as the p-PLL to triolein ratio), plasmid DNA started to escape from the complex and free DNA band (Lane 7) appeared in the agarose gel.

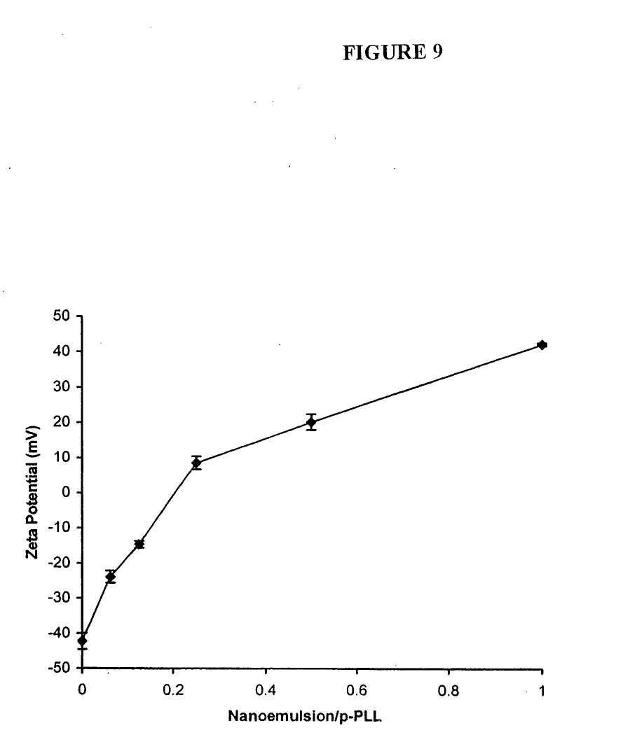

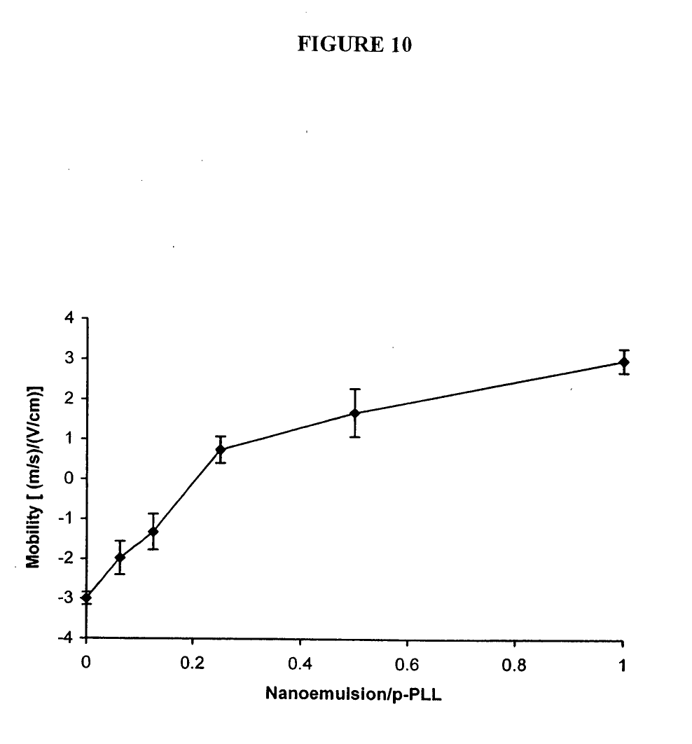

Since the surface charge of the nanoemulsion/p-PLL/DNA complex is very important to transfection, the zeta potential and mobility of these complexes were measured and the results are shown in FIG. 9 and FIG. 10. With fixed amount of plasmid DNA (2 μg), the increased amount of p-PLL led to an increase in zeta potential of the particles.

Transfection of Glioma Cell Line SF-767 by the Complex of Nanoemulsion/p-PLL/DNA

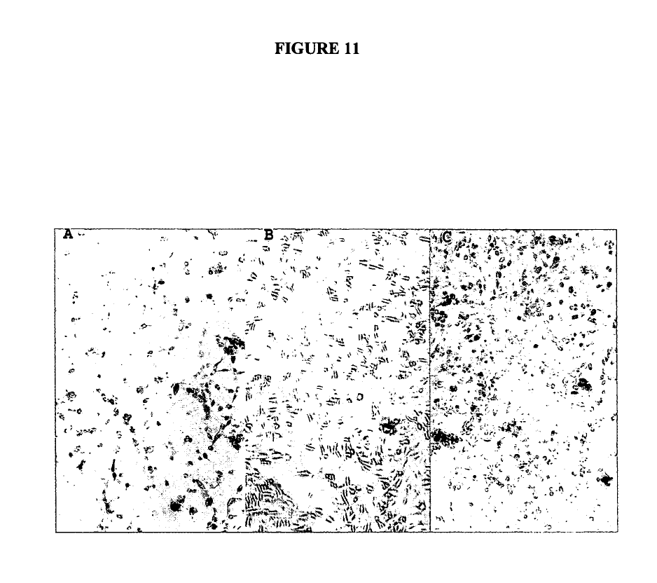

Most of the positively charged nanoemulsion/p-PLL/DNA complexes (with varying ratios of nanoemulsion/p-PLL/DNA) used in the experiments were found to transfect the glioma SF-767 cells, but with different transfection efficiency. The complex containing nanoemulsion and p-PLL (p-PLL:triolein=0.25:1) and 2 μg DNA, which had a zeta potential of 8.47±1.85 mV and a loading capacity of 1 μg DNA per 0.25 mg of lipid (or per 50 μl of formulation), had the highest transfection efficiency. Its efficiency was comparable to that by Lipofectamine™ reagent (FIG. 11). Under microscope, those cells that expressed active β-galactosidase appeared to be blue-green (as dark spots in FIG. 6) by X-Gal staining and the extent of transfection appeared to be comparable for the nanoemulsion complex (FIG. 11, C) to that by Lipofectamine™ reagent (FIG. 11, A). However, Lipofectamine™ reagent appeared to be much more toxic than the nanoemulsion complex, as indicated by the significant difference in cell counts.

Effect of Chloroquine on the Transfection

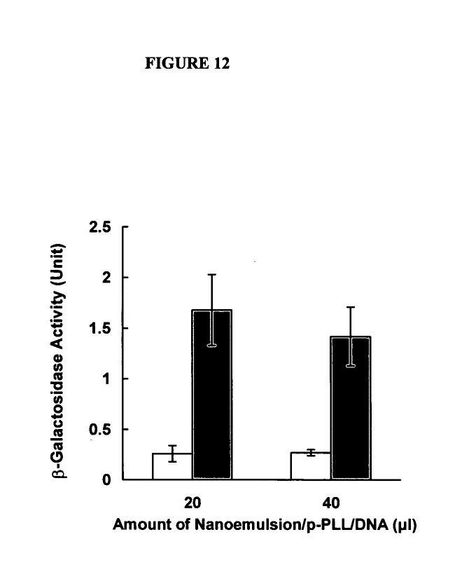

Cellular uptake of particles via endocytosis will result in the particles being processed by endosomal-lysosomal pathway. This pathway will lead to the degradation of the carried plasmid DNA and greatly lower the transfection efficiency. Chloroquine is a weak basic, lysomotropic drug, which will interfere with the endosomal acidification and cause the bursting of endosomes. In order to examine whether the nanoemulsion vector is delivered via endosomal-lysosomal pathway, cells were treated with 200 μM of chloroquine at 37° C. for 30 minutes before the transfection procedures was conducted. The β-galactosidase activity of the cells with or without the treatment of chloroquine was shown in FIG. 12, based on two quantities of the nanoemulsion/p-PLL/DNA complexes. The treatment of the cells by chloroquine solution greatly increased the transfection efficiency and the effect was obvious at both complex quantities. This result suggested that endocytosis be the main mechanism of the cellular uptake of the nanoemulsion/p-PLL/DNA complex by glioma cells.

Cellular Toxicity Evaluation of Nanoemulsion/p-PLL Complex

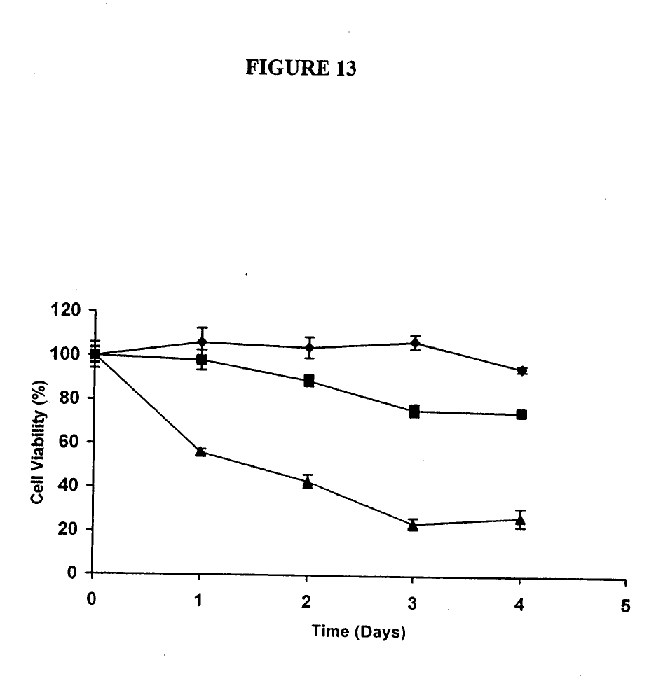

Cellular toxicity is one of the main concerns in the development of gene delivery system. It has been commonly shown that positively charged gene delivery systems, such as cationic liposomes, are cytotoxic. Other cationic polymers, e.g. poly-L-lysine, hydrophobized poly-L-lysine, were also found cytotoxic (21,25). Since the nanoemulsion particles in this research were negatively charged, the incorporation of positively charged and lipidized poly-L-lysine resulted in a neutralization of the charges on the surface. The cytotoxicity of the nanoemulsion/p-PLL complex, in comparison with that of the commercial Lipofectamine™ reagent, was shown in FIG. 13. The cytotoxicity was evaluated based on the relative viability of cells grown on the EMEM medium with and without the delivery systems. Four days after transfection, cell culture supplemented with the nanoemulsion/p-PLL complex had 75% cellular viability while that supplemented by Lipofectamine™ reagent (which is cationic liposome) had only 24% cell viability.

Discussion

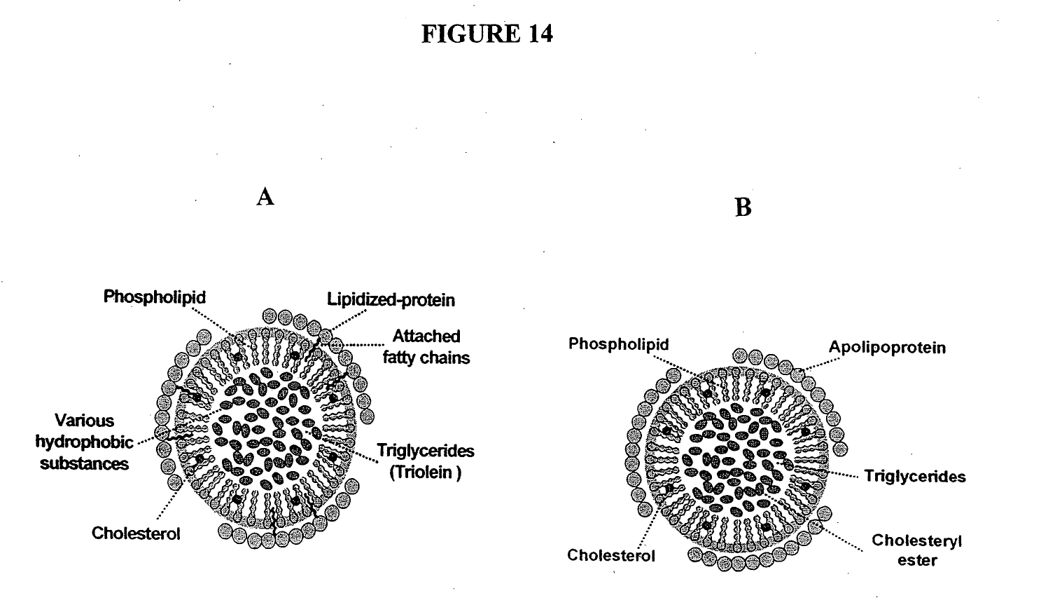

Effective gene transfection depends on the ability of the carrier system to deliver gene to and transfect in specific cells with high transfection efficiency and low cytotoxicity. Many synthetic carrier systems have been investigated with certain success and most of them belong to the category of cationic liposomes. However, DNA/liposome complex is in general cytotoxic and thus less cytotoxic but efficient gene carriers have been investigated. One of such approaches is to develop gene carriers that somewhat mimic the natural carriers in human body. Kim et al. (21, 22) developed a novel terplex system based on the natural low-density lipoprotein associated with hydrophobized poly-L-lysine. The system is capable of condensing DNA and subsequently transfecting cells. Hara et al. (26) described the use of reconstituted chylomicrons remnants (RCR) as a non-viral vector for gene delivery. DNA was complexed with cationic lipid and solubilized in the core of these RCR particles. Both of these lipoprotein-based systems appeared to offer advantages over the conventional cationic liposome systems. Recently, our laboratory has attempted to develop an artificial lipoprotein system for controlled drug delivery. The artificial lipoprotein, similar to natural lipoprotein, consists of a phospholipid nanoemulsion particles with functional proteins attached on the particle surfaces. A schematic drawing of the artificial lipoprotein system can be seen in FIG. 14, in comparison with natural human lipoproteins. Based on our earlier work in lipoprotein-resembling nanoemulsion for controlled delivery of an anti-tumor cholesteryl carborane compound (27), this paper describes a new attempt to develop an artificial lipoprotein system that has poly-L-lysine attached on the particle surfaces for the purpose of gene delivery. The cytotoxicity of such a system has been specifically investigated in comparison with commercial Lipofectamine™ reagent.

The lipoprotein-resembling particles were made of commercially available lipids and lipidized poly-L-lysine. The lipidization of poly-L-lysine was achieved through N-alkylation of the free ε-amino groups with palmitoyl chloride and confirmed via proton NMR (data not shown). The reaction condition was controlled to only lipidize about 25% of the lysine residues preserving sufficient amount of free ε-amino groups for maintaining the ability of poly-L-lysine to condense DNA. The nanoemulsion particles carried negative surface charge as shown by agarose gel electrophoresis in FIG. 7. When nanoemulsion particles are combined with unlipidized poly-L-lysine, immediate precipitation was observed indicating the formation of large aggregates due to charge neutralization. When appropriate amount of p-PLL was incubated with the particles, no precipitation or change in the turbidity was observed. These results indicate that with lipidized poly-L-lysine the interaction was not merely through electrostactic interaction, but also through hydrophobic interaction between the palmitoyl chains of p-PLL and the phospholipid of nanoemulsion particles. The charge of the nanoemulsion particles became reversed when sufficient amount of p-PLL was added (FIG. 7).

The surface charge of the complex formed by nanoemulstion and p-PLL depends on their relative ratio. In order to carry DNA molecules, which are negatively charged, the carrier needs to be positive. On the other hand, the surface charge of the complex after DNA is incorporated is also critical. Since cell surface is negatively charged, a positively charged nanoemulsion/p-PLL/DNA complex is essential for successful transfection. The surface charge of these particles can be monitored by agarose gel electrophoresis qualitatively or by zeta potential and mobility measurement quantitatively (FIG. 8, 9 and 10). Our studies showed that most of the positively charged complex formed by varying the ratios of nanoemulsion, p-PLL and DNA can transfect the glioma cells in certain extent. The complex containing nanoemulsion, p-PLL and DNA with the zeta potential of 8.47±1.85 mV achieved the highest transfection efficiency indicating the proper charge balance among these components was important for transfection. This observation is consistent with that reported by Kim et al. (21) when the terplex carrier system involving natural LDL was employed for gene delivery.

Chloroquine has been widely used to investigate the cellular uptake mechanism (28-29). It will interact with endosome inside the cell. A positive correlation of chloroquine level with the transfection efficiency indicates that the DNA is taken up through endocytosis and, furthermore, the endosomal-lysosomal pathway. Through chloroquine treatment, we have shown that the endocytosis appears the major cellular uptake pathway for the lipoprotein-resembling gene carrier.

Cytotoxicity is an important consideration for developing novel gene delivery systems. Using new gene carriers that mimic the nature substance such as human lipoproteins, we can significantly reduce the cytotoxicity associated with the delivery systems. It is known that poly-L-lysine is very toxic to cells (Morgan, 1989). Its complex with the phospholipid nanoemulsion particles and plamid DNA, however, has low cytotoxic as indicated by our experiments in comparison with the Lipofectamine™ system. The nature of phospholipids, the neutralization of the positive charge of poly-L-lysine and the proper balance among nanoemulsion, p-PLL and DNA apparently contribute to the reduced cytotoxicity for this new gene delivery system.

In conclusion, the novel artificial lipoprotein system of the present invention may be used in vitro gene transfection to mammalian cells, especially tumor cells. The system mimics the natural lipoprotein in composition but contains lipidized poly-L-lysine (instead of surface protein) to carry genetic materials. Such a system can be conveniently formulated from natural lipids, with the ability to control the size and surface charge. With proper ratios among its components, the new gene delivery system shows a similar transfection efficiency but a lower cytotoxicity compared with the commercial Lipofectamine™ gene transfection system, making it especially useful as an alternative to these commercial gene transfection systems.

### Further Example—Transfection of DNA-Delivery of DNA Vaccine

Our previous work has not examined any therapeutic gene, especially the DNA vaccine, carried by the artificial lipoprotein. DNA vaccines expressing rabies virus glycoprotein have been constructed and tested for immunogenicity and protection against challenge. Although these constructs are capable of inducing a protective immunity, large amount of the DNA is required. Furthermore, it takes more than six weeks to develop measurable responses. Thus, it is necessary to improve the efficiency of the DNA vaccines. In this work, we used artificial lipoproteins as a carrier to deliver a DNA vaccine expressing rabies virus glycoprotein into cell culture. Similar to the structure of natural lipoproteins, the artificial lipoprotein delivery system consists of nanoemulsion cores made of natural lipids and surface lipidized poly-L-lysine, which replaces the surface protein as in natural lipoprotein. The gene encoding rabies virus glycoprotein was constructed in plasmid and used as the rabies DNA vaccine. The surface charge parameters (zeta potential and mobility) of the artificial lipoprotein/plasmid DNA complex was determined. The effect of varying the ratio of lipidized poly-L-lysine to the nanoemulsion on transfection efficiency (expressing the rabies virus glycoprotein in human glioma cell line SF767) was studied. In addition, a comparison study of the capability of transfecting human glioma SF-767 cell line between the artificial lipoprotein and Lipofectamine™ 2000 was conducted.

### EXPERIMENTAL PROCEDURE

### Preparation of Nanoemulsion and Synthesis of Palmitoyl poly-L-lysine

In brief, the lipid components of oily phase (triolein, 70%; egg yolk phosphatidylcholine, 22.7%; lysophosphatidylcholine, 2.3%; cholesterol oleate, 3%; and cholesterol, 2%) were dissolved separately in chloroform and mixed thoroughly. Organic solvent was removed and NaCl solution (2.4 M) was added to dried lipids in ratio 0.01:1 (w/v, total weight of lipids in gram vs. ml of 2.4 M NaCl solution). The mixture was sonicated using a Model 450 Sonifier (Branson Ultrasonics Corporation, Danbury, Conn.) and passed through an Emulsiflex B3 device (Avestin, Ontario, Canada). Submicron particle Sizer Model 370 (Nicomp Particle Sizing System, San Barbra, Calif.) was used to measure particle size distribution of nanoemulsion.

Lipidization of Poly-L-Lysine (PLL) was performed as follows. Palmitoyl fatty chain was linked to the free amino group of PLL. The resulting polymer, palmitoyl poly-L-lysine (p-PLL) was verified by H-NMR (d-DMSO) and characterized by agarose gel electrophoresis using Nile Red as the fluorescent dye (Nile Red stains the palmitoyl chains). Agarose gel (0.4%) was prepared in TAE buffer (40 mM Tris-acetic acid, 1 mM EDTA, pH 8.0). Twenty μl of PLL solution (1 mg/ml) and 20 μl of p-PLL solution (1 mg/ml) were added to dried Nile Red dye and incubated for 30 min, respectively. Six μl of glycerin was added to increase the density of the sample and the samples were then loaded into the wells of the agarose gel. Horizontal mini-gel system (CBS Scientific Company Inc, Calif., USA) was used to run the electrophoresis for one hour at 90 volts (20 mA) at room temperature. The mobility of the samples in the electric field was visualized by ChemiImager™ System (Alpha Innotech Corporation, San Leandro, Calif.).

Assembly of Nanoemulsion, p-PLL and Rabies Plasnid DNA Complex

Fifty μl of NE was diluted with 0.2 ml phosphate buffered saline (PBS) solution. Various amounts of p-PLL were incubated for one hour at 37° C. with diluted NE based on the weight ratio of p-PLL to triolein (triolein was a major component of NE). The weight ratios were 0:1, 0.125:1, 0.25:1, 0.5:1, 1:1 and 2:1 (p-PLL to triolein). After the incubation, 2 μg of pCDNA-RG (rabies glycoprotein plasmid DNA) was added and incubated for 15 minutes at room temperature. The electrophoretic mobility of NE/p-PLL and NE/p-PLL/pCDNA-RG were examined using agarose gel (0.4%) as described above. Zeta potential and mobility of the NE/p-PLL/pCDNA-RG complexes were measured by Submicron Particle Size Analyzer 9OPlus (Brookhaven Instrument Corporation, Holtsville, N.Y., USA).

Evaluation of Transfection Efficiency

For transfection, NE (50 μl) was diluted with 0.2 ml PBS solution and incubated with various amount of p-PLL (p-PLL:triolein ratio of 0.125:1, 0.25:1, 0.5:1, and 1:1) for one hour at 37° C. After the incubation, 2 μg of pCDNA-RG was added and incubated for 15 minutes at room temperature. One ml of EMEM (without FBS) was added to each well following by addition of NE/p-PLL/pCDNA-RG complex and incubated for 12 hr at 37° C. and 5% CO₂. Afterward, incubation media was discarded and one ml of EMEM containing 20% FBS was added to each well and incubated for additional 36 hr. The transfected cells were rinsed three times with PBS and then fixed in acetone solution (80% v/v, acetone:water) for 10 minute at −20° C. Fixed cells were air-dried and stored in refrigerator pending indirect immunofluorescence assay (IFA). For IFA, cells were rinsed three times with PBS and a 1:50 dilution of a rabies glycoprotein-specific mouse monoclonal anti-G-antibody (Accurate Chemical and Scientific Corporation) was added Cells were incubated for 1 hr at 37° C. and rinsed three times with PBS (3-4 minutes each). A 1:100 dilution of a secondary goat anti-mouse fluorescein (FITC)-conjugated antibody (Accurate Chemical and Scientific Corporation) was added and cells were incubated for 1 hr at 37° C. Cells were rinsed three times with PBS, air-dried, and examined using an Olympus fluorescence microscopy (Olympus American Inc, Melville, N.Y.).

Lipofectamine 2000 was used as the positive control. The ratios of DNA to lipofectamine (w/v) were 1:0.5, 1:2, 1:3 and 1:5 according to the manufacture protocol for optimization of the transfection. NE/p-PLL/pCDNA-3 (plasmid only without rabies gene) was used as the negative control. In addition, p-PLL/pCDNA-RG (similar amount of p-PLL but with no nanoemulsion) was also used as the formulation control.

The transfection efficiency of transient expression of rabies glycoprotein in the glioma cells was determined by flow cytometric evaluation. Binding of rabies glycoprotein-specific mouse monoclonal anti-G-antibody was revealed by secondary staining using secondary goat anti-mouse fluorescein (FITC)-conjugated antibody. The staining procedure was similar to that for the above-mentioned microscopic examination but with some modifications. Since flow cytometry runs cells in suspension, the cells were washed and suspended in PBS. Suspended cells were incubated with the first antibody (1:50 dilution) for 1 hr. Cells were centrifuged, media was discarded, and cells washed with PBS. Cells were incubated with the secondary antibody (1:100) for 1 hr, centrifuged, washed with PBS, and resuspended in PBS. Twenty thousand cells for each sample were examined in FACS Calibur flow cytometry device (Becton Dickimson, San Jose, Calif.) using FLOWJO software (Tree Star Inc, San Carlos, Calif.). Clumps and debris were excluded using forward and side-scatter windows. Transfection efficiency was calculated based on the percentage of the positive cells (that expressed rabies glycoprotein) in total number of cells.

Results and Conclusion DNA Vaccine Experiment Described Above

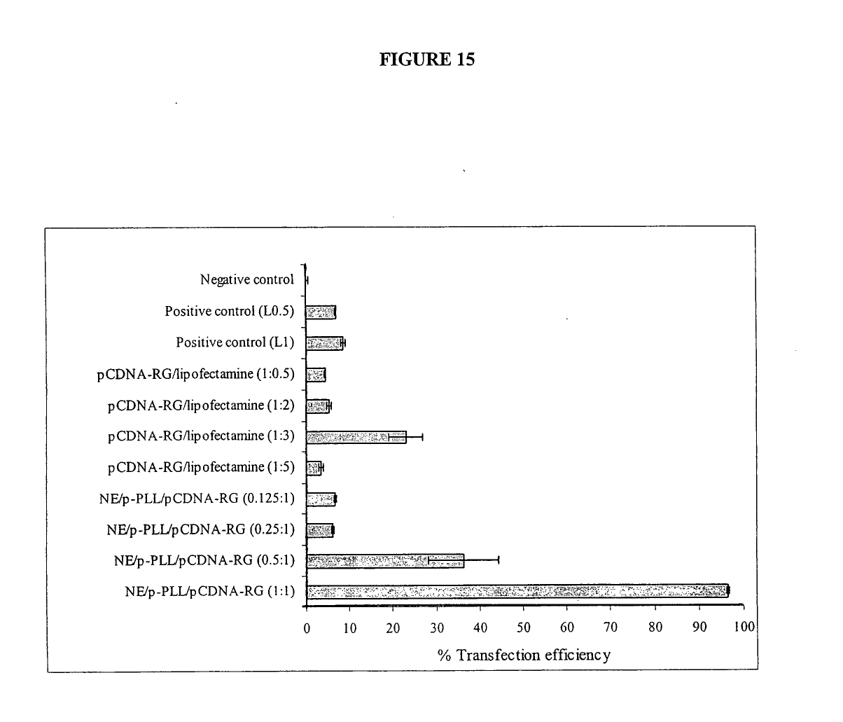

The expression of rabies glycoprotein was monitored by using antibody against the rabies virus glycoprotein. The transfected cells were fixed with acetone and reacted with polyclonal antibodies against rabies virus glycoprotein, followed by FITC-conjugated anti-mouse secondary antibody. The expression of rabies virus glycoprotein was then visualized under fluorescence microscopy. Binding of FITC antibody with glycoprotein on the cell membrane of transfected cells made the cells show light green fluorescence. The non-transfected cells, however, lacked of antibody specific affinity (i.e. absence of rabies glycoprotein) and exhibited a black background. The transfection can be clearly observed from the cells that expressed rabies glycoprotein and were tagged with the fluorescent probe. Transfection efficiency of the rabies glycoprotein plasmid DNA carried by the artificial lipoprotein formulation and lipofectamine 2000 in human glioma SF-767 cell line is presented in FIG. 15. The transfection efficiency of the negative control (NE/p-PLL/pCDNA-3) was 0.30±0.23. The transfection efficiency of the positive controls (p-PLL/pCDNA-RG) was 6.86±0.1 (L0.5) and 8.67±0.51(L1), respectively. The transfection efficiency of rabies DNA carried by lipofectamine 2000 was 4.46±0.23, 5.39±0.43, 23±3.85 and 3.5±0.59 when the ratio of pCDNA-RG/lipofectamine was formulated as 1:0.5, 1:2, 1:3 and 1:5, respectively. However, the transfection efficiency of rabies DNA carried by our artificial lipoprotein system (NE/p-PLL/pCDNA-RG complex) was 6.76±0.32, 6.25±0.21, 36.33±8.08 and 96.30±0.14 for the formulations with p-PLL:triolein ratio of 0.125:1, 0.25:1, 0.5:1, and 1:1, respectively. The transfection using the artificial lipoprotein system was effective and the transfection efficiency of the new system was significantly higher than that of lipofectamine 2000.

The highest transfection efficiency was achieved when the cells were incubated with the artificial lipoprotein complex composing of NE/p-PLL/pCDNA-RG at 1:1 p-PLL:triolein ratio, when the amount of pCDNA-RG was kept constant at 2 μg/50 μl of NE. The % TE of the negative control was detected at very low level (0.30±0.23), indicating the sensitivity of the detection method. Increase of p-PLL amount in the artificial lipoprotein formulation resulted in an increase in the % TE. This phenomenon could be attributed to the increase in net surface positivity of the NE/p-PLL/pCDNA-RG complex, resulting in a greater affinity to interact with the negatively charged cell membrane. The NE/p-PLL/pCDNA-RG complexes (p-PLL:triolein ratios of 0.5:1, and 1:1) showed up to 5.3 fold and 11 fold increase in the % TE compared to p-PLL/pCDNA-RG complex when equivalent amounts of p-PLL and pCDNA-RG were used, respectively. The results clearly demonstrated the important role of the artificial lipoprotein carrier system. The highest % TE with lipofectamine 2000 was 23±3.85 and it was achieved with 1:3 ratio (pCDNA-RG:lipofectamine) according to the manufacture recommended protocol. Compared to the lipofectamine system, the artificial lipoprotein carrier system for DNA illustrated about 4.2 fold increase in % TE based on the highest % TE, indicating the high effectiveness of the new carrier system.

By way of the example described above, a novel artificial lipoprotein system has been developed as a new carrier for DNA vaccines. The amount of p-PLL incorporated into the artificial lipoprotein formulations had a significant effect on transfection efficiency. The new system demonstrated a highly effective transfection capability of rabies DNA vaccine in cell culture. The new system also showed to be more efficient in cellular transfection of rabies DNA vaccine than the commercial lipofectamine 2000 formulation.

### REFERENCES

1. S. Lehrman. Virus treatment questioned after gene therapy death, Nature 401:517-518 (1999).

2. E. Marshall. Gene therapy death prompts review of adenovirus vector, Science 286: 2244-2245 (1999).

3. W. T. Godbey, A. G. Mikos. Recent progress in gene delivery using non-viral transfer complexes, J. Control. Release 72:115-125 (2001).

4. F. Liu, L. Huang. Development of non-viral vectors for systemic gene delivery, J. Control. Release 78:259-266 (2002).

5. S. Han, R. I. Mahato, Y. K. Sung, S. W. Kim. Development of biomaterials for gene therapy, Molecular Therapy 2(4):302-317 (2000).

6. X. Gao, L. Huang. Potentiation of cationic liposome-mediated gene delivery by polycations, Biochemistry 35(3): 1027-1036 (1996).

7. X. Zhou, L. Huang. DNA transfection mediated by cationic liposomes containing lipopolylysine: characterization and mechanism of action, Biochim. Biophys. Acta 1189(2):195-203 (1994).

8. J. Zabner, A. J. Fasbender, T. Moninger, K. A. Poellinger, M. J. Welsh. Cellular and molecular barriers to gene transfer by a cationic lipid, J. Biol. Chem. 270:18997-19007 (1995).

9. Y. Xu, F. C. Szoka Jr. Mechanism of DNA release from cationic liposome/DNA complexes used in cell transfection, Biochemistry 35(18):5616-5623 (1996).

10. O. Zelphati, F. C. Szoka Jr. Mechanism of oligonucleotide release from cationic liposomes, Proc. Natl. Acad. Sci. USA 93(21):11493-11498 (1996).

11. Y. Tan, F. Liu, Z. Li, L. Huang. Sequential injection of cationic liposome and plasmid DNA delivery gene effectively to the lung with minimal inflammatory toxicity, Mol. Ther. 3(5):673-682 (2001).

12. H. Lee, J. H. Jeong, T. G. Park. A new gene delivery formulation of polyethylenimine/DNA complexes coated with PEG conjugated fusogenic peptide, J. Control. Release 76:183-192 (2001).

13. W. Guo, R. J. Lee. Efficient gene delivery via non-covalent complexes of folic acid and polyethylenimine, J. Control. Release 77:131-138 (2001).

14. W. T. Godbey, K. K. Wu, A. G. Mikos. Poly(ethylenimine) and its role in gene delivery, J. Control. Release 60(2-3):149-160 (1999).

15. D. Fischer, T. Bieber, Y. Li, H. P. Elsasser, T. Kissel. A novel non-viral vector for DNA delivery based on low molecular weight, branched polyethylenimine: effect of molecular weight on transfection efficiency and cytotoxicity, Pharm. Res. 16(8):1273-1279 (1999).

16. D. Putnam, R. Langer. Poly(4-hydroxy-L-proline ester): low-temperature polycondensation and plasmid DNA complexation, Macromolecules 32:3658-3662 (1999).

17. K. W. Leong, H. Q. Mao, V. L. Truong-Le, K. Roy, S. M. Walsh, J. T. August. DNA-polycation nanospheres as non-viral gene delivery vehicles, J. Control. Release 53(1-3):183-193 (1998).

18. R. Puls, R. Minchin. Gene transfer and expression of a non-viral polycation-based vector in CD4+ cells, Gene Ther. 6(10):1174-1178 (1999).

19. D. Oupicky, C. Konak, K. Ulbrich, M. A. Wolfert, L. W. Seymour. DNA delivery system based on complexes of DNA with synthetic polycations and their copolymers, J. Control. Release 65(1-2):149-171 (2000).

20. H. Gonzalez, S. J. Hwang, M. E. Davis. New class of polymers for the delivery of macromolecular therapeutics, Bioconjug. Chem. 10(6):1068-1074 (1999).

21. J.-S. Kim, A. Maruyama, T. Akaike, S. W. Kim. In vitro gene expression on smooth muscle cells using a terplex delivery system, J. Control. Release 47:51-59 (1997).

22. J.-S. Kim, B.-I. Kim, A. Maruyama, T. Akaike, S. W. Kim. A new non-viral DNA delivery vector: the terplex system, J. Control. Release 53:175-182 (1998).

23. M. M. Bradford. A rapid and sensitive method for the quantitation of microgram quantities of protein utilizing the principle of protein-dye binding, Anal. Biochem. 72:248-254 (1976).

24. T. Mosmann. Rapid colorimetric assay for cellular growth and survival: application to proliferation and cytotoxicity assays, J. Immunol. Methods 65:55-63 (1983).

25. D. M. L. Morgan, V. L. Lavin, and J. D. Pearson. Biochemical characterization of polycation-induced cytotoxicity to human vascular endothelial cells, J. Cell. Sci. 94:553-559 (1989).

26. T. Hara, Y. Tan, and L. Huang. In vivo gene delivery to the liver using reconstituted chylomicron remnants as a novel nonviral vector, Proc. Natl. Acad. Sci. USA 94:14547-14552 (1997).

27. M. Shawer, P. Greenspan, S. Oie and D. R. Lu. VLDL-resembling phospholipid-submicron emulsion for cholesterol-based drug targeting, J. Pharm. Sci. 91:1405-1413 (2002).

28. H. Luthman, and G. Magnusson. High efficiency polyoma DNA transfection of chloroquine treated cells, Nucl. Acids Res. 11:1295-1308 (1983).

29. C.-W. Cho, Y.-S. Cho, H.-K. Lee, Y. II Yeom, S.-N. Park, and Y. Yoon. Improvement of receptor-mediated gene deliver to HepG2 cells using an amphiphilic gelling agent, Biotech. Appl. Biochem. 32:21-26 (2000).

It is to be understood by those skilled in the art that the foregoing description and examples are illustrative of practicing the present invention, but are in no way limiting. Variations of the detail presented herein may be made without departing from the spirit and scope of the present invention as defined by the following claims.

1-33. (canceled)

34. A pharmaceutical composition comprising:
i. a microemulsion comprising a lipid core and an amphipathic lipid layer surrounding said lipid core comprising positively charged lipids, wherein a hydrophobic portion of said amphipathic lipid layer is associated with said lipid core and a hydrophilic portion of said amphipathic lipid layer is associated with a hydrophilic surface of said amphipathic lipid said amphipathic lipid;
ii. a polynucleotide; and
iii. optionally, a lipidized polymer;
said amphipathic lipid being included in said composition in an amount effective to form a microemulsion with said lipid core, and said lipidized polymer being included in said composition in association with said amphipathic lipid in an amount ranging from about 0.01% to about 65% by weight of said composition.

35. The composition according to claim 34 wherein said polynucleotide is associated with said positively charged lipid at the surface of said microemulsion.

36. The composition according to claim 34 wherein said amphipathic lipid includes an amount of a steroid component effective to increase the stability of said amphipathic lipid.

37. The composition according to claim 34 wherein said amphipathic lipid comprises a phospholipid.

38. The composition according to claim 34 wherein said phospholipid is selected from the group consisting of phosphatidylcholine, cephalin, isolecithin, phosphatidylethanolamine, distearoylphosphatidylcholine, phosphatidylserine, phosphatidylglycerol, phosphatidic acid, phosphatidylinositol, sphingomyelin, dimyristoylphosphatidylcholine, dimyristoylphosphatidylglycerol, pegylated phospholipids and mixtures, thereof.

39. The composition according to claim 37 wherein said amphipathic lipid layer further comprises about 0.05% to about 25% by weight of a steroidal component.

40. The composition according to claim 39 wherein said steroidal component is selected from the group consisting of cholesterol, pegylated cholesterol, coprostanol, cholestanol, cholestane, C₁, to C₂₄ steroidal esters and mixtures, thereof.

41. The composition according to claim 34 wherein said polynucleotide is DNA.

42. The composition according to claim 34 wherein said lipidized polymer is a lipidized protein or polypeptide.

43. The composition according to claim 42 wherein said lipidized polypeptide is lipidized polylysine.

44. The composition according to claim 42 wherein said protein is selected from the group consisting of enzymes, cell surface proteins, hormones, antibodies, growth factors, clotting factors, neuroproteins, tumor suppressors, toxins, antigens and epitopes of antigens, apolipoproteins, endogenous or exogenous tumor antigenic proteins, bioinvasive molecules (like bacterial invasins), lectins, lectin-like molecules, bacterial toxins such as cholera and macromolecules with bioadhesive properties.

45. The composition according to claim 44 wherein said protein is selected from immunoglobulins, epitopes, transferrin, avidin, hormones, enzymes, integrin, apolipoprotein E, apolipoprotein B100, and mixtures, thereof.

46. The composition according to claim 44 wherein said protein is lysozyme, avidin, apolipoprotein B100 or apolipoprotein E.

47. The composition according to claim 34 wherein said lipid core comprises a mono-, di- or triglyceride.

48. The composition according to claim 47 wherein said lipid core comprises triglycerides.

49. The composition according to claim 48 wherein said triglyceride is triolein.

50. A method of enhancing the delivery of a polynucleotide to a predetermined site or tissue within a subject comprising administering to said patient a composition according to any of claim 34 to said subject.

51. A method of enhancing the delivery of a polynucleotide to a predetermined site or tissue within a subject comprising administering to said patient a composition according to any of claim 35 to said subject.

52. A method of enhancing the delivery of a polynucleotide to a predetermined site or tissue within a subject comprising administering to said patient a composition according to claim 37 to said subject.

53. A method of enhancing the delivery of a polynucleotide to a predetermined site or tissue within a subject comprising administering to said patient a composition according to claim 41 to said subject.

54. The method according to claim 50 wherein said DNA is naked DNA.

55. The method according to claim 51 said DNA is naked DNA.

56. The method according to claim 53,said DNA is naked DNA.
